(19) United States
(12) Patent Application Publication
(10) Pub. No.: US 2023/0364234 A1
(43) Pub. Date: Nov. 16, 2023

(54) COMPOSITIONS AND METHODS FOR CHIMERIC ANTIGEN RECEPTOR (CAR)-MODIFIED CELL MODULATION

(71) Applicant: ALBERT EINSTEIN COLLEGE OF MEDICINE, Bronx, NY (US)

(72) Inventors: Harris GOLDSTEIN, Bronx, NY (US); Steven C ALMO, Bronx, NY (US); Scott GARFORTH, Bronx, NY (US); Mengyan LI, Bronx, NY (US)

(21) Appl. No.: 17/905,323

(22) PCT Filed: Mar. 8, 2021
(86) PCT No.: PCT/US2021/021407

§ 371 Date: Jul. 27, 2023

Related U.S. Application Data
(60) Provisional application No. 62/986,574, filed on Mar. 6, 2020.

(51) Int. Cl. A61K 39/00; C07K 16/28; A61K 39/395

(52) U.S. Cl. CPC A61K 39/4631; C07K 16/2818; C07K 16/2878; A61K 39/3955; C07K 2319/41; C07K 2317/622; C07K 2319/33; A61K 2239/39; A61K 2239/48

(57) ABSTRACT

Polypeptides, nucleic acids, and compositions thereof are provided that include a targeting moiety. The polypeptides, nucleic acids, and compositions that comprise them, can be used in methods to treat subjects, to alter CAR-expressing cells in subjects who may suffer from a disease such as a cancer or a pathogen.

### CROSS-REFERENCE TO RELATED APPLICATION

This application claims priority to U.S. Provisional Application No. 62/986,574 filed on Mar. 6, 2020, the disclosure of which is hereby incorporated by reference herein in its entirety.

### STATEMENT OF GOVERNMENT SUPPORT

This invention was made with government support under AI145024 awarded by the National Institute of Allergy and Infectious Disease (NIAID) and CA198095 awarded by the National Cancer Institute (NCI). The government has certain rights in the invention.

### SEQUENCE LISTING

This disclosure includes a sequence listing, which was submitted in ASCII format via EFS-Web, and is hereby incorporated by reference in its entirety. The ASCII copy, created on Mar. 4, 2021, is named Sequence_Listing_8001WO00. and is 60 kilobytes in size.

### BACKGROUND

Chimeric antigen receptor (CAR) expressing T-cells (CAR-T cells) or CAR-expressing natural killer (NK) cells are a therapeutic approach that enables the mobilization of the immune response to treat previously refractory B-cell malignancies. In CAR-T cells, instead of the standard T cell receptor (TCR) MHC-restricted antigen recognition molecule expressed by T cells, CAR-T cells express an WIC-independent recombinant CAR consisting of an extracellular antibody-derived or ligand-derived binding fragment and an intracellular domain which can include TCR zeta chain activation domains and costimulatory signaling domains such as CD28 and/or 4-1BBL. For example, the antibody-derived binding fragment binds a target antigen such as CD19, a molecule expressed by B cells, resulting in activation of the CAR-T cell via signal transduction triggered by the intracellular signaling domains of the CAR. After recognition of CD19 expressed by the B cells, these CD19-specific CAR-T cells will eliminate the targeted B cells, including malignant B cells which cause leukemia and lymphoma. This approach has revolutionized therapy for pre-B cell acute lymphoblastic leukemia (ALL) and B cell lymphomas by providing a new strategy to eliminate malignant B cells and cure patients with ALL or B cell lymphoma refractory to previous treatments. CAR-expressing cells can also be engineered to treat infectious diseases. For example, CARs can be engineered to recognize HIV antigens expressed on the surface of infected cells, in an MHC-independent manner, and stimulate the CAR-T cell through intracellular signaling domains that include TCR activation and costimulatory domains to kill the HIV-infected cells.

First-generation HIV-specific CAR-T cells transferred into HIV-infected individuals displayed long-term persistence but no significant clinical effect. Subsequently, the ex vivo activity of HIV-specific CAR-T cells was markedly improved by using enhanced immunoreceptors costimulatory signaling domains and by enhancing the resistance of the CAR-T cells to HIV infection by ablating expression of CCR5, the primary HIV coreceptor. In addition to its established efficacy for the treatment of malignancies, these improvements have also made CAR-T cells an attractive therapeutic modality to contribute to the functional cure of HIV infection. However, analyses of long-term outcomes of CAR-T cell treatments for cancer have revealed that the disease remission induced by CAR-T cell therapy frequently does not persist due to poor maintenance of CAR-T cell function or the emergence of resistant cancer cells. Furthermore, off-target activity or overly enhanced immune activation by CAR-T cells may cause toxicity requiring supportive therapies and would benefit from the rapid inhibition of CAR-T cell activity. However, at present, once CAR-T cells are infused into patients, there is no available strategy to specifically amplify or inhibit their activities. In addition, the costimulatory signals required for T cell activation, expansion, and function in current CAR-T cells are provided by integrating the costimulatory signaling domains into the CAR construct, which limits modulation of CAR-T cell function to the small number (e.g., 1 or 2) costimulatory domains directly integrated into the CAR construct. Developing a method to specifically deliver additional endogenous modulatory signals (i.e., not encoded within the CAR) to CAR-T cells after infusion would provide the full spectrum of T cell functionality to enable the in vivo reactivation and amplification of their capacity to eliminate target cells, or to suppress their activities if the CAR-T cells are mediating toxic effects. These endogenous functions include, but are not limited to, costimulatory signals (e.g., ICOS, CD40L, OX40, CD27 and GITR), coinhibitory signals (e.g., PD-1, CTLA-4 and TIM3) and/or a variety of cytokine signals (e.g., IL-2, IL-7, IL-12 and IL-15).

For this purpose, there is a need to develop novel biologics that specifically modulate in vivo and ex vivo activity of infused malignancy- or pathogen-specific CAR-expressing cells (e.g., CAR-T cells or CAR-expressing natural killer (NK) cells).

### SUMMARY

In one aspect, the disclosure relates to a fusion polypeptide comprising a targeting moiety, wherein the targeting moiety recognizes a targeted moiety, and an additional moiety, wherein the additional moiety recognizes a modulatory domain on the cell.

In certain embodiments, the targeting moiety may further recognizes a targeted epitope selected from the group consisting of a myc epitope, a FLAG epitope, a histidine (His) epitope, a hemagglutinin (HA) epitope, a glutathione-s transferase (GST) epitope, a TAP epitope, a V5 epitope, calmodulin binding protein (CBP) epitope, a maltose binding protein (MBP) epitope, or a fluorescent epitope.

In certain embodiments, the targeting moiety may be an antibody, fragment, or derivative thereof.

In certain embodiments the targeting moiety is located at an N-terminal end of the fusion polypeptide.

In certain embodiments, the fusion polypeptide may include no major histocompatibility complex (MHC) polypeptide sequence, variant, or fragment thereof.

In certain embodiments, the modulatory domain is a co-stimulatory ligand, a cytokine, a co-inhibitory ligand, or any combination thereof.

In certain embodiments, the modulatory domain may include a polypeptide sequence that is at least a portion of the polypeptide sequence of a CD28 polypeptide, a 4-1BBL polypeptide, a GITR polypeptide, CD27 polypeptide, a B7-1 polypeptide, a B7-2 polypeptide, an ICOS-L polypeptide, an OX-40L polypeptide, a CD80 polypeptide, a CD86 polypeptide, a PD-L1 polypeptide, a FasL polypeptide, and a PD-L2 polypeptide.

In certain embodiments, the modulatory domain may include a polypeptide sequence that is at least a portion of the polypeptide sequence of CD7, CD30L, CD40, CD70, CD83, HLA-G, MICA, MICB, HVEM, lymphotoxin beta receptor, 3/TR6, ILT3, and ILT4.

In another aspect, the disclosure relates to a fusion polypeptide comprising a single chain variable (scFv) domain, wherein the scFv domain includes: at least one targeting domain that targets a portion of a myc epitope; and a CD28 polypeptide antibody, fragment, or derivative thereof or a 4-1BBL polypeptide antibody, fragment, or derivative thereof.

In certain embodiments, compositions comprising fusion polypeptides are provided.

In certain embodiments, compositions may further include a chimeric antigen receptor (CAR)-expressing cell, wherein the CAR-expressing cell comprises a targeted moiety that is recognized by the targeting moiety of the fusion polypeptide.

In certain embodiments, compositions may further include a pharmaceutically acceptable excipient.

In certain embodiments, fusion polypeptides or compositions provided herein may be suitable for use in in treating and/or preventing a disease or condition in a subject.

In certain embodiments, fusion polypeptides or compositions provided herein may be for use in the production of a medicament.

In another aspect, a nucleic acid comprising a nucleotide sequence encoding a fusion polypeptide is provided. In certain embodiments, the fusion polypeptide comprises a single chain variable (scFv) domain, wherein the scFv domain includes at least one targeting moiety.

In certain embodiments, the at least one targeting moiety targets at least a portion of a myc epitope, a FLAG epitope, a histidine (His) epitope, a hemagglutinin (HA) epitope, a glutathione-s transferase (GST) epitope, a TAP epitope, a V5 epitope, calmodulin binding protein (CBP) epitope, a maltose binding protein (MBP) epitope, or a fluorescent epitope.

In certain embodiments, the targeting moiety is an antibody, fragment, or derivative thereof.

In certain embodiments, the nucleic acid includes the at least one targeting moiety located at a 5′ end of the nucleic acid.

In certain embodiments, the nucleic acid may further include a modulatory domain.

In certain embodiments, the modulatory domain is a co-stimulatory ligand, a cytokine, a co-inhibitory ligand, or any combination thereof.

In certain embodiments, the modulatory domain comprises a polypeptide sequence that is at least a portion of the polypeptide sequence of a CD28 polypeptide, a 4-1BBL polypeptide, a GITR polypeptide, CD27 polypeptide, a B7-1 polypeptide, a B7-2 polypeptide, an ICOS-L polypeptide, an OX-40L polypeptide, a CD80 polypeptide, a CD86 polypeptide, a PD-L1 polypeptide, a FasL polypeptide, and a PD-L2 polypeptide.

The nucleic acid of claim 19, wherein the modulatory domain is selected from CD7, CD30L, CD40, CD70, CD83, HLA-G, MICA, MICB, HVEM, lymphotoxin beta receptor, 3/TR6, ILT3, and ILT4.

In another aspect, a nucleic acid comprising a nucleotide sequence encoding a fusion polypeptide is provided. The fusion polypeptide having a single chain variable (scFv) domain, wherein the scFv domain includes: at least one targeting domain that targets a portion of a myc epitope; and a CD28 polypeptide antibody, fragment, or derivative thereof or a 4-1BBL polypeptide antibody, fragment, or derivative thereof.

In certain embodiments, compositions are provided comprising a nucleic acid according to the present disclosure.

In certain embodiments, the compositions may further include a pharmaceutically acceptable excipient.

In another aspect, an in vivo method of modulating a CAR-expressing cell is provided, the method comprising administering to a subject the subject a fusion polypeptide according to the present disclosure or a composition according to the present disclosure, wherein the CAR-expressing cell comprises a targeted moiety that is recognized by the targeting moiety of the fusion polypeptide.

In certain embodiments, the modulation of the CAR-expressing cell induces proliferation of the CAR-expressing cell, induces differentiation of the CAR-expressing cell, stimulates the CAR-expressing cell, inhibits the CAR-expressing cell, or any combination thereof.

In certain embodiments, the modulation of the CAR-expressing cell is via direct targeting of the CAR-expressing cell.

In certain embodiments, the modulation of the CAR-expressing cell is at least about 0.5-fold higher as compared to modulation of a CAR-expressing cell without the epitope that is recognized by the targeting domain of the fusion polypeptide.

In another aspect, a method of treating a subject is provided, the method comprising administering to the subject an amount of a fusion polypeptide according to the present disclosure or a composition according to the present disclosure.

In certain embodiments, the subject is further administered a CAR-expressing cell, wherein the CAR-expressing cell comprises a targeted moiety that is recognized by the targeting moiety of the fusion polypeptide.

In certain embodiments, the CAR-expressing cell is administered prior to, subsequent to, or concurrently with the fusion polypeptide.

In certain embodiments, the subject has a disease associated with expression of a tumor antigen or a disease associated with a pathogen.

In certain embodiments, the disease associated with expression of a tumor antigen is a proliferative disease, a precancerous condition, a cancer, or a non-cancer related indication associated with expression of the tumor antigen.

In certain embodiments, the cancer is chronic lymphocytic leukemia (CLL), acute leukemia, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia.

In certain embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of the cancers, and metastatic lesions of the cancers.

In certain embodiments, the cancer is leukemia or lymphoma.

In certain embodiments, the lymphoma is lymphoblastic lymphoma or B-cell Non-Hodgkin's lymphoma.

In certain embodiments, the pathogen is a viral pathogen.

In certain embodiments, the viral pathogen is Human immunodeficiency virus (HIV).

In certain embodiments, the method of treating the subject comprises modulation of the CAR-expressing cell.

In certain embodiments, modulation of the CAR-expressing cell induces proliferation of the CAR-expressing cell, induces differentiation of the CAR-expressing cell, stimulates the CAR-expressing cell, inhibits the CAR-expressing cell, or any combination thereof.

In certain embodiments, the modulation of the CAR-expressing cell is via direct targeting of the CAR-expressing cell.

In certain embodiments, the modulation of the CAR-expressing cell is at least about 0.5-fold higher as compared to modulation of a CAR-expressing cell without the targeted moiety that is recognized by the targeting domain of the fusion polypeptide.

In certain embodiments, the subject experiences an improvement in an inflammatory response, a cytokine storm, or at least one off-target effect, or any combination thereof as compared to a control subject.

In certain embodiments, the subject experiences an improvement in a symptom or a side effect as compared to a control subject.

In certain embodiments, the symptom is cytokine-release syndrome (CRS), a neurologic toxicity, B-cell aplasia, tumor lysis syndrome (TLS), anaphylaxis, fever, joint/muscle aches, shortness of breath, low blood pressure, confusion, a seizure.

This listing is intended to be exemplary and illustrative rather than comprehensive and limiting. Additional aspects and embodiments may be set out in, or apparent from, the remainder of this disclosure and the claims.

### DESCRIPTION OF THE DRAWINGS

The accompanying drawings exemplify certain aspects and embodiments of the present disclosure. The depictions in the drawings are intended to provide illustrative, and schematic rather than comprehensive, examples of certain aspects and embodiments of the present disclosure. The drawings are not intended to be limiting or binding to any particular theory or model and are not necessarily to scale. Without limiting the foregoing, nucleic acids and polypeptides may be depicted as linear sequences, or as schematic, two- or three-dimensional structures; these depictions are intended to be illustrative, rather than limiting or binding to any particular model or theory regarding their structure.

FIG. 1 is a schematic depiction of the composition of the present disclosure.

FIG. 1A: The fusion polypeptide contains a targeting moiety (α-tag VH and VL) which is specific for a targeted moiety such as a myc epitope incorporated into the CAR and a binder (COSTIM) for a co-stimulatory ligand (COSTIM-R) expressed on the CAR-expressing cell (depicted here is a CAR-T cell). FIG. 1B: The fusion polypeptide incorporates a targeted moiety such as a myc epitope that is recognized by a targeting moiety (α-tag VH and VL) incorporated into the CAR and a binder (COSTIM) specific for a co-stimulatory ligand (COSTIM-R) expressed on the CAR-expressing cell and.

FIG. 2 is a schematic depiction of HIV-gp120-specific CAR-T cell activation by a fusion polypeptide as disclosed herein, where the fusion polypeptide is specific for a myc epitope incorporated into the CAR and co-stimulatory ligands expressed on the CAR-T cell.

FIGS. 3A-3B are schematic depictions of fusion polypeptides as disclosed herein. FIG. 3A depicts a fusion polypeptide linked to α-CD28 to activate the CD28 costimulatory pathway on target CAR-T cells. FIG. 3B depicts a fusion polypeptide linked to 4-1BBL co-stimulatory agonists to activate the 4-1BB co-stimulatory pathway on target CAR-T cells.

FIGS. 4A-4C illustrate a CAR-T cell-myc epitope lentivector. Expression of the myc epitope enables the CAR to be targeted by a fusion polypeptide as disclosed herein. FIG. 4A is a schematic depiction of an original SFFV/GFP lentivirus construct and a construct engineered to express the CD4-10-17b CAR-T cell vector with an inserted myc epitope. FIG. 4B is a schematic depiction of an original CD4-10-17b CAR molecule (left panel) and a CAR molecule with an inserted myc epitope (right panel). FIG. 4C shows representative flow cytometry data for Jurkat/MA cells transduced with lentiviral vectors SFFV/GFP (left panels) or SFFV/CAR-T myc/GFP (right panels) stained with anti-myc (clone:9E10) and evaluated for GFP expression (top panels) or CD4 expression (lower panels).

FIGS. 5A-5B demonstrate detection of myc-epitope in primary lentivector-transduced T cells. Purified primary T cells were transduced with the control lentivirus (upper panels) or with the myc epitope-expressing CAR lentivector (lower panels). FIG. 5A shows the fraction of cells binding the 9E10 anti-myc antibody after gating on CD4⁺GFP⁺ T cells. FIG. 5B shows the fraction of cells binding the 9E10 anti-myc antibody after gating on CD8⁺GFP⁺ T cells.

FIG. 6 demonstrates that fusion polypeptides as disclosed herein stimulated interferon gamma (IFN-γ) production by CAR-T cells expressing CD4⁺. T cells transduced with a negative control lentiviral vector control (SFFV/GFP empty vector) or T cells transduced with a myc epitope-expressing CAR-T lentivector were stimulated overnight as indicated. The T cells were analyzed by flow cytometry gated for GFP and CD4 expression and IFN-γ expression was determined.

### DETAILED DESCRIPTION

### Definitions and Abbreviations

Unless otherwise specified, each of the following terms has the meaning set forth in this section.

The indefinite articles “a” and “an” denote at least one of the associated noun and are used interchangeably with the terms “at least one” and “one or more.” For example, the phrase “a module” means at least one module, or one or more modules.

The conjunctions “or” and “and/or” are used interchangeably.

“Domain” is used to describe a segment of a protein or nucleic acid. Unless otherwise indicated, a domain is not required to have any specific functional property.

“Subject” means a human, mouse, or non-human primate. A human subject can be any age (e.g., an infant, child, young adult, or adult), and may suffer from a disease, such as a cancer.

“Treat,” “treating,” and “treatment” as used herein mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” as used herein means the prevention of a disease in a subject, e.g., in a human, including (a) avoiding or precluding the disease; (b) affecting the predisposition toward the disease; (c) preventing or delaying the onset of at least one symptom of the disease.

The terms “polynucleotide,” “nucleotide sequence,” “nucleic acid,” “nucleic acid molecule,” “nucleic acid sequence,” and “oligonucleotide” refer to a series of nucleotide bases (also called “nucleotides”) in DNA and RNA and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. A nucleotide sequence typically carries genetic information, including the information used by cellular machinery to make proteins and enzymes. These terms include double- or single-stranded genomic DNA, RNA, any synthetic and genetically manipulated polynucleotide, and both sense and antisense polynucleotides. This also includes nucleic acids containing modified bases.

Conventional IUPAC notation is used in nucleotide sequences presented herein, as shown in Table 1, below (see also Cornish-Bowden 1985, incorporated by reference herein). It should be noted, however, that “T” denotes “Thymine or Uracil” insofar as a given sequence (such as a gRNA sequence) may be encoded by either DNA or RNA.

TABLE 1
IUPAC nucleic acid notation

| Character | Base |
|---|---|
| A | Adenine |
| T | Thymine |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G, or T/U |
| V | A, C, or G |
| H | A, C, or T/U |
| D | A, G, or T/U |
| N | A, C, G, or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments, variants, derivatives and analogs of such proteins. Peptide sequences are presented using conventional notation, beginning with the amino or N-terminus on the left, and proceeding to the carboxyl or C-terminus on the right. Standard one-letter or three-letter abbreviations may be used.

The term “antigen” as used herein refers to an immunogenic molecule, subunit, or fragment thereof capable of provoking an immune response. This immune response may involve activation of specific immunologically-competent cells, antibody production, or both. An antigen may be, for example, a peptide, glycopeptide, polypeptide, glycopolypeptide, polysaccharide, lipid, or the like. For example, an antigen may be any molecule or a linear molecular fragment such as a peptide, which is derived by processing of the native antigen that can be recognized by a T-cell receptor (TCR). DNA sequences encoding an antigen may be provided. The DNA sequences that encode an antigen can be optimized for expression in a cell. For example, the DNA sequences encoding an antigen may further comprise a promoter upstream of the DNA sequence encoding the antigen, or other heterologous DNA sequences, a transcription termination signal downstream of the sequence encoding the antigen, or both. It will be apparent to one of skill in the art that an antigen can be produced recombinantly, synthesized, or derived from a biological sample. Exemplary biological samples that can contain one or more antigens include, for example, biological fluids, cells, tumor samples, tissue samples, or combinations thereof. Antigens can be produced by one or more cells that have been genetically engineered or modified to express an antigen. The antigens may be optimized for stable transcription or expression by the one or more cells that have been genetically engineered or modified to produce the antigen. For example, the antigens may be codon optimized by techniques including, but not limited to, by altering sequences of four of the same nucleotides in a row (e.g., AAAA, TTTT, GGGG, CCCC) by introducing point mutations that do not result in amino acid changes in the encoded protein, or by adapting codon usage for use in a specific cell species.

The term “epitope” as used herein includes any structure, molecule, amino acid sequence, or protein determinant that is recognized and specifically bound by a cognate binding molecule, for example, a T cell receptor, chimeric antigen receptor, or any other binding domain, molecule, or protein.

The term “single chain variable” or its abbreviation “scFv” as used herein refers to a single-chain variable fragment of an antibody. An scFv is a fusion protein of the variable regions of the heavy and light chains of immunoglobulins, connected by a short linker peptide.

The term “chimeric antigen receptor” (CAR) refers to a CAR of the present disclosure engineered to contain two or more naturally occurring (or engineered) amino acid sequences linked together in a way that does not occur naturally or does not occur naturally in a host cell, which CAR can function as a receptor when present on a surface of a cell. CARs of the present disclosure include an extracellular portion comprising an antigen-binding domain, such as one obtained or derived from an immunoglobulin, such as an scFv derived from an antibody linked to a transmembrane region and one or more intracellular signaling domains (optionally containing co-stimulatory domain(s)) (see, e.g., Sadelain et al., 2013; see also Harris & Kranz, 2016; Stone et al., 2014).

A “T cell” or “T lymphocyte” is an immune system cell that matures in the thymus and produces TCRs, including αβT cells and γδT cells. T cells can be naïve (not exposed to antigen; increased expression of CD62L, CCR7, CD28, CD3, CD127, and CD45RA, and decreased expression of CD45RO as compared to TCM), memory T cells (TM) (antigen-experienced and long-lived), and effector cells (antigen-experienced, cytotoxic). TM can be further divided into subsets of central memory T cells (TCM, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or TCM).

A “natural killer cell” or “NK cell” is an immune system cell, a type of cytotoxic lymphocyte, that is capable of rapidly responding to a wide variety of pathological challenges. NK cells are capable of killing virus-infected cells and detecting and killing stressed cells and tumor cells without requiring any priming or prior activation, i.e., they do not require antibodies and a major histocompatibility complex (MHC) to be presented on the cell surface to kill a cell. This ability allows NK cells to respond faster than other immune cells such as cytotoxic T cells.

A peripheral blood mononuclear cell (PBMC) is a diverse class of immune cells that includes lymphocytes (T cells, B cells, and NK cells), dendritic cells, and monocytes. PBMCs originate from hematopoietic stem cells (HSCs) in the bone marrow and give rise to all of the cells in the immune system. PBMCs refer to any peripheral blood cell that has a round nucleus.

An “MHC protein” as used herein refers to a major histocompatibility complex (MHC) protein. MHC proteins are also called human leukocyte antigens (HLA), or the H2 locus in mice, which are protein molecules that are expressed on the surface of a cell that confer a unique antigenic identity to the cell. MHC/HLA antigens serve as target molecules that are recognized by T-cells and NK cells as being derived from the same source of hematopoietic reconstituting stem cells as the immune effector cells (“self”) or as being derived from a different source of hematopoietic reconstituting cells (“non-self”). Two primary classes of HLA antigens are recognized (HLA class I and HLA class II). The MHC proteins used herein may be from any mammalian or avian species, for example, primates (particularly humans); rodents (including mice, rats, and hamsters); equines, bovines, canines, felines, rabbits etc. Of particular interest are the human HLA proteins and the murine H-2 proteins. Included in the HLA proteins are the class I proteins HLA-A, HLA-B, HLA-C, and β2-microglobulin, and the class II subunits HLA-DPα, HLA-DPβ, HLA-DQα, HLA-DQβ, HLA-DRα and HLA-DRβ. Included in the murine H-2 subunits are the class I H-2K, H-2D, H-2L, and the class II I-Aα, I-Aβ, I-Eα and I-Eβ, and β2-microglobulin. The MHC binding domains are typically a soluble form of the usually membrane-bound protein. The soluble form is derived from the native form by a deletion of the transmembrane domain. Conveniently, the protein is truncated, removing both the cytoplasmic and transmembrane domains. In certain embodiments, the binding domains of an MHC protein are soluble domains of class II α and β chain. In some such embodiments, the binding domains have been subjected to mutagenesis and selected for amino acid changes that enhance the solubility of the single chain polypeptide without altering the peptide binding contacts.

A “T cell receptor” or “TCR” as used herein refers to the antigen/MHC binding heterodimeric protein product of a vertebrate, e.g. mammalian, TCR gene complex, including the human TCR α, β, γ, and δ chains. A TCR can be recombinant, such as a protein product that is not derived from a mammalian TCR gene complex, or naturally-occurring (e.g., derived from a mammalian TCR gene complex).

All antigens, proteins, peptides, targeting moieties, targeted moieties, targets nucleic acids, modulatory domains, and the like recited herein may have at least about 70%, at least about 75%, at least about 80%, at least about 85%, at least about 90%, at least about 95%, at least about 98%, at least about 99%, or 100% sequence homology with the listed sequence, gene, or protein.

### Overview

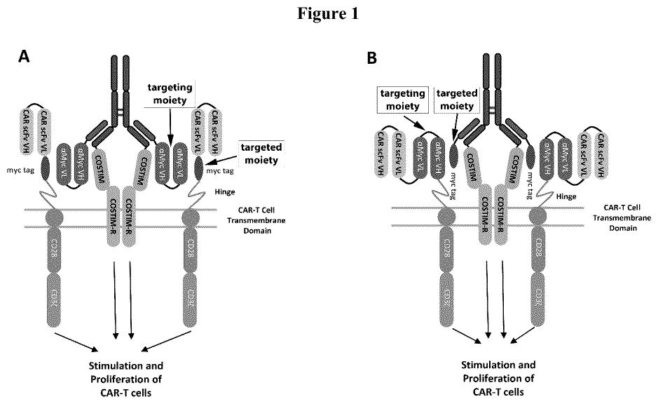

In certain aspects, the present disclosure relates to novel fusion polypeptides designed to selectively bind chimeric antigen receptor (CAR) modified cells (e.g., CAR-T cells) and modulate the function of said cells either ex vivo or after infusion into patients. The compositions and methods disclosed herein may utilize a targeting moiety which targets (i.e., specifically recognizes) a targeted moiety, wherein interaction between the targeting moiety and the targeted moiety directs selective binding between a fusion polypeptide and a CAR-expressing cell (e.g., CAR-T cell; see FIG. 1). In some embodiments, the targeting moiety is provided on the fusion polypeptide and the targeted moiety is provided on the CAR-expressing cell (e.g., CAR-T cell; see FIG. 1A). In other embodiments, the targeting moiety is provided on the CAR-expressing cell and the targeted moiety is provided on the fusion polypeptide (FIG. 1B).

In certain embodiments provided herein, novel fusion polypeptides may be designed to selectively bind CAR-expressing cells by recognizing a unique epitope contained within the CAR expressed by the CAR-expressing cell or a targeted moiety incorporated into the CAR. In certain embodiments, a fusion polypeptide may possess a targeted moiety which is recognized by a targeting moiety expressed by a CAR-expressing cell either as a component of the CAR or an additional molecule co-expressed with the CAR on CAR-expressing cells. For example, the targeting moiety may be co-expressed with the CAR vector using a 2A peptide linker or an Internal Ribosomal Entry Site (“IRES”). In some embodiments, a fusion polypeptide may also include an additional moiety, the additional moiety selectively binds one or more modulatory domains on the surface of the CAR-expressing cell. Binding of the additional moiety to the one or more modulatory domains on the surface of the CAR-expressing cell may be used to regulate CAR-expressing cell function. The modulatory domain may be a naturally-occurring modulatory domain, or it may be a modulatory domain that the cell is engineered to express. Exemplary modulatory domains suitable for use with the present disclosure include, but are not limited to, CD28, Inducible Costimulator (ICOS, also referred to as CD278), CD134 (OX40, TNFRSF4), CD137 (TNFRSF9), CD27 (TNFRSF7), GITR (TNFRSF18), CD154 (CD40 ligand, TNFSF5), CD152 (CTLA-4), CD279 (PD-1), FcγRIII (CD16), killer inhibitory receptors (KIR), lectin-like receptor CD94, NKG2, NKp30, NKp44, NKp46, 2B4, DNAM-1, or the like. The one or more modulatory domains may be a positive modulatory domain (e.g., upon interaction with a corresponding additional moiety results in increased function, activation, expansion, or the like) or a negative modulatory domain (e.g., upon interaction with a corresponding additional moiety results in decreased function, inhibition, suppression, apoptosis, or the like). For example, binding to a positive modulatory domain on the CAR-expressing cell surface may provide a co-stimulatory signal (antigen non-specific signal provided by interaction between co-stimulatory molecules on an immune cell and an antigen presenting cell) and/or cytokine signal to increase CAR-expressing cell function (i.e., by in vivo activation, expansion, and differentiation), thereby augmenting the CAR-expressing cells' ability to eliminate malignant or infected cells. Alternatively, by binding to a negative modulatory domain, the additional moiety may provide an inhibitory signal to selectively suppress or delete a specific CAR-expressing cell population.

In other embodiments, a targeted moiety and a targeting moiety are utilized to direct selective binding of CAR-expressing cells by the disclosed fusion polypeptides. In certain embodiments, the targeted moiety is expressed by the CAR-expressing cell and the targeting moiety is on the fusion polypeptide. In other embodiments, the targeted moiety is on the fusion polypeptide and the targeting moiety is expressed by the CAR-expressing cell. For example, the CAR-expressing cell may be modified so that, in addition to expressing a CAR, a CAR-expressing cell also expresses the targeted moiety to direct binding of the disclosed fusion polypeptides comprising a targeting moiety to a CAR-expressing cell by engaging a targeted moiety on the CAR or a targeted moiety expressed on the CAR-expressing cell surface, for example, by adding a nucleic acid sequence encoding a targeted moiety or gene comprising a targeted moiety to the CAR vector. In certain embodiments, the targeted moiety may be a myc epitope, a FLAG epitope, a His epitope, a HA epitope, a GST epitope, a TAP epitope, a V5 epitope, a CBP epitope, an MBP epitope, a fluorescent epitope, or the like. The present technology can be used with any CAR so long as there is a unique epitope to use as the targeted moiety, such as an epitope located at an scFv/hinge or an scFv/transmembrane junction, or the like. In one embodiment, the targeted moiety is incorporated into a CAR construct such that cells transduced with the construct express a CAR that comprises the targeted moiety. In another embodiment, the targeted moiety is expressed on the CAR-expressing cell surface. In certain embodiments, the targeted moiety may be co-expressed with the CAR vector using a 2A peptide linker or an IRES. In another embodiment, the targeted moiety is incorporated into the fusion polypeptide to direct binding to a targeting moiety expressed by the CAR-expressing cell, for example, on the CAR-expressing cell surface. In both embodiments, incorporation of the targeted moiety into the CAR-expressing cell or fusion polypeptide directs selective binding of CAR-expressing cells by the disclosed fusion polypeptides. As discussed above, in embodiments where the targeted moiety is on the CAR-expressing cell, the targeted moiety may be on the CAR or on the CAR-expressing cell surface (e.g., by incorporating the targeted moiety into a CAR construct, or co-expressing the targeted moiety using a 2A peptide linker or IRES).

### Fusion Polypeptides

The present disclosure provides bispecific fusion polypeptides that selectively bind to a CAR-expressing cell (via interaction between a targeting moiety and a targeted moiety) and one or more modulatory domains on a CAR-expressing cell surface. As discussed above in the Overview, the targeted moiety may be provided on the fusion polypeptide or it may be expressed by the CAR-expressing cell (see, e.g., FIGS. 1A, 1B). In certain embodiments, the fusion polypeptides comprise a targeting moiety recognizes a targeted moiety on the CAR-expressing cell (either on a CAR or the CAR-T cell surface) which allows the fusion polypeptide to selectively bind to the targeted moiety. These fusion polypeptides may further comprise an additional moiety that selectively binds to the one or more modulatory domains on the CAR-expressing cell surface (see, e.g., FIG. 1A). Binding of the fusion polypeptide to the one or more modulatory domains results in delivery of costimulatory and/or cytokine signals or inhibitory signals via the additional moiety to selectively modulate CAR-expressing cell function. The modulatory domain may be a naturally-occurring modulatory domain, or it may be a modulatory domain that the cell is engineered to express. Exemplary modulatory domains suitable for use with the present disclosure include, but are not limited to, CD28, Inducible Costimulator (ICOS, also referred to as CD278), CD134 (OX40, TNFRSF4), CD137 (TNFRSF9), CD27 (TNFRSF7), GITR (TNFRSF18), CD154 (CD40 ligand, TNFSF5), CD152 (CTLA-4), CD279 (PD-1), FcγRIII (CD16), killer inhibitory receptors (KIR), lectin-like receptor CD94, NKG2, NKp30, NKp44, NKp46, 2B4, DNAM-1, or the like. The one or more modulatory domains may be a positive modulatory domain (e.g., upon interaction with a corresponding additional moiety results in increased function, activation, expansion, or the like) or a negative modulatory domain (e.g., upon interaction with a corresponding additional moiety results in decreased function, exhaustion, inhibition, suppression, apoptosis, or the like).

In certain embodiments, the fusion polypeptides provided herein comprise a targeting moiety that selectively binds to one or more targeted moieties comprised within a CAR. Selective binding of the fusion polypeptides to CAR-expressing cells expressing CARs with the targeted moiety provides a mechanism for selective delivery of costimulatory and/or cytokine or inhibitory signals—only those CAR-expressing cells comprising the targeted moiety will be modulated.

Targeting/targeted moieties of the present disclosure may comprise any suitable pair of binding moieties including, but not limited to, proteins, lipids, drugs, or chemicals. Examples of suitable targeted moieties that may be provided as part of a fusion polypeptide, a CAR, or a CAR-expressing cell surface include, but are not limited to, a myc epitope (e.g., EQKLISEEDL (SEQ ID NO: 1)), a FLAG epitope (e.g., DYKDDDK (SEQ ID NO: 2)), a histidine (His) epitope (e.g., His5 (SEQ ID NO: 3) or His6 (SEQ ID NO: 4)), a hemagglutinin (HA) epitope (e.g., YPYDVPDYA (SEQ ID NO: 5)), a glutathione-s transferase (GST) epitope, a TAP epitope, a V5 epitope, a calmodulin binding protein (CBP) epitope, a maltose binding protein (MBP) epitope, or a fluorescent epitope. The targeted moiety may also be any protein or other compound for which a ligand exists that binds to it with high affinity which can function as the targeting moiety such as biotin-binding proteins, lipid binding proteins, bacterial periplasmic binding proteins, lectins, serum albumins, immunoglobulins, inactivated enzymes, insect pheromone-binding proteins, immunosuppressant-binding proteins, phosphate-binding proteins, and sulfate-binding proteins (de Wolf and Bret Pharmacological Reviews 2000:52:207-236).

In certain embodiments, the fusion polypeptides, CARs, or CAR-expressing cell surfaces provided herein comprise a targeting moiety wherein the targeting moiety comprises an antibody sequence (i.e., a complete immunoglobulin light and/or heavy chain) or a fragment or derivative thereof. For example, the fusion polypeptides, CARs, or CAR-T cell surfaces provided herein may comprise an antibody sequence or a fragment or derivative thereof from an antibody that specifically binds a myc epitope (e.g., 9E10 clone, 9E10.3 clone, 27H46L35 clone, and 9E11 clone), a FLAG epitope (e.g., 20H18L16, 20H1L23, 8H2L5, 8H8L17, 8HCLC, L5, and FG4R), a His epitope (e.g., 4E3D10H2/E3, HIS.H8, 4A12E4, and 21HCLC), an HA epitope (e.g., HA.C5, 4C12, 16B12, and HA-7), a GST epitope, a TAP epitope, a V5 epitope, a CBP epitope, an MBP epitope, or a fluorescent epitope. In some embodiments, the antibody sequence is derived from a monoclonal antibody. In other embodiments, the antibody sequence is derived from a polyclonal or oligoclonal antibody. In some embodiments, the antibody sequence is fully or partially humanized.

In certain embodiments wherein the fusion polypeptides, CARs, or CAR-expressing cells provided herein comprise a targeting moiety comprising an antibody sequence or a fragment or derivative thereof, the targeting moiety may comprise one or more single chain variable fragments (scFvs). Thus, in certain embodiments the fusion polypeptides, CARs, or CAR-expressing cell surfaces provided herein comprise at least one scFv. In other embodiments, the fusion polypeptides, CARs, or CAR-expressing cell surfaces comprise at least 2, at least 3, at least 4, at least 5, at least 6, at least 7, at least 8, at least 9, at least 10, or more scFv domains.

In certain embodiments, each scFv comprises one targeting moiety, and in these embodiments each scFv binds to only one targeted moiety. In other embodiments, each scFv may comprise 2, 3, 4, 5, 6, 7, 8, 9, 10, or more than 10 targeting moieties. In these embodiments, a single scFv may bind more than one targeted moiety. In some embodiments, the scFvs may comprise less than 10, less than 9, less than 8, less than 7, less than 6, less than 5, less than 4, less than 3, or less than 2 targeting moieties.

In certain embodiments, the scFv comprises a targeting moiety that is specific for an antigen that is present on the surface of a tumor cell or an infected cell. In some embodiments, the scFv is specific for CD19, CD22, gp120, ROR1, or FGFR4.

In certain embodiments, the fusion polypeptides, CARs, or CAR-expressing cells provided herein may comprise one or more targeted moieties wherein the targeted moiety is a tag. Exemplary tags include, but are not limited to, horseradish peroxidase (HRP), biotin, a fluorescent tag, FITC, or phycoerythrin.

The scFv targeting moiety in the fusion polypeptide can be located at any position within a fusion polypeptide, a CAR, or a CAR-T cell surface, including bound to a heavy or light immunoglobulin chain. In some embodiments, the targeting moiety is located at an N-terminal end of the fusion polypeptide or CAR. In other embodiments, the targeting moiety is located at a C-terminal end of the fusion polypeptide or CAR. In yet other embodiments, the targeting moiety is located in the middle or substantially the middle of the fusion polypeptide or CAR, or in a non-scFv domain of the fusion polypeptide or CAR.

In certain embodiments, the fusion polypeptides, CARs, or CAR-expressing cells provided herein may comprise one or more targeted moieties wherein the targeted moiety is a tag. Exemplary tags include, but are not limited to, horseradish peroxidase (“HRP”), biotin, a fluorescent tag, FITC, or phycoerythrin.

In other embodiments, a CAR vector is designed to express both a CAR and a targeting moiety that specifically targets a corresponding targeted moiety. In this embodiment, the fusion polypeptide comprises the corresponding targeted moiety (FIG. 1B). For example, in this embodiment, the CAR vector may express both a CAR and a targeting moiety, wherein the targeting moiety comprises a scFv or ligand specific to a corresponding targeted moiety, wherein the targeted moiety is a myc epitope, and wherein the fusion polypeptide comprises the corresponding targeted moiety, in this case a myc epitope. In this embodiment, the targeted moiety-specific scFv or ligand may be expressed on the surface of a CAR-expressing and may specifically recruit the fusion polypeptide by binding the targeted moiety incorporated in the fusion polypeptide, thereby allowing the fusion polypeptide to deliver a primary, costimulatory, inhibitory, and/or cytokine signal.

In some embodiments, the fusion polypeptide further comprises at least 1 modulatory domain. The modulatory domains of the present disclosure function as a costimulatory signal, a cytokine, and/or an inhibitory signal to modulate CAR-expressing cells. In some embodiments, the fusion polypeptide further comprises at least 2, at least 3, at least 4, at least 5, at least 6, at least 7, at least 9, at least 10, or more modulatory domains. In some embodiments, the fusion polypeptide comprises less than 10, less than 9, less than 8, less than 7, less than 6, less than 5, less than 4, less than 3, or less than 2 modulatory domains.

In some embodiments, the at least one modulatory domain is selected from an anti-CD28 polypeptide such as the bispecific scFv set forth in SEQ ID NO: 6, a 4-1BBL polypeptide such as the single chain trimer (“SCT”) set forth in SEQ ID NO:7, a GITR polypeptide such as the polypeptide set forth in SEQ ID NO: 8, CD27 polypeptide such as the polypeptide set forth in SEQ ID NO: 9, a B7-1 polypeptide (also referred to as CD80) such as the polypeptide set forth in SEQ ID NO: 10, a B7-2 polypeptide (also referred to as CD86) such as the polypeptide set forth in SEQ ID NO: 11, an ICOS-L polypeptide such as the polypeptide set forth in SEQ ID NO: 12, an OX-40L polypeptide such as the polypeptide set forth in SEQ ID NO: 13, a PD-L1 polypeptide such as the polypeptide set forth in SEQ ID NO: 14, a FasL polypeptide such as the polypeptide set forth in SEQ ID NO: 15, and a PD-L2 polypeptide such as the polypeptide set forth in SEQ ID NO: 16, or antibodies, fragments, or derivatives thereof recognizing the cognate ligands for these modulatory domains.

In some embodiments, the at least one modulatory domain is an anti-CD28 polypeptide and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 6.

In some embodiments, the at least one modulatory domain is a 4-1BBL polypeptide and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 7.

In some embodiments, the at least one modulatory domain is a GITR polypeptide and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 8.

In some embodiments, the at least one modulatory domain is a CD27 polypeptide and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 9.

In some embodiments, the at least one modulatory domain is a B7-1 polypeptide (also referred to as CD80) and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 10.

In some embodiments, the at least one modulatory domain is a B7-2 polypeptide (also referred to as CD86) and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 11.

In some embodiments, the at least one modulatory domain is a an ICOS-L polypeptide and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 12.

In some embodiments, the at least one modulatory domain is an OX-40L polypeptide and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 13.

In some embodiments, the at least one modulatory domain is a PD-L1 polypeptide and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 14.

In some embodiments, the at least one modulatory domain is a FasL polypeptide and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 15.

In some embodiments, the at least one modulatory domain is a PD-L2 polypeptide and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 16.

In some embodiments, the at least one modulatory domain is selected from CD7 (SEQ ID NO: 17), CD30L (SEQ ID NO: 18), CD40 (SEQ ID NO: 19), CD70 (SEQ ID NO: 20), CD83 (SEQ ID NO: 21), HLA-G (SEQ ID NO: 22), MICA (SEQ ID NO: 23), MICB (SEQ ID NO: 24), HVEM (SEQ ID NO: 25), lymphotoxin beta receptor (SEQ ID NO: 26), 3/TR6 (SEQ ID NO: 27), ILT3 (SEQ ID NO: 28), ILT4 (SEQ ID NO: 29) or antibodies, fragments, or derivatives thereof recognizing the cognate ligands for these modulatory domains.

In some embodiments, the at least one modulatory domain is CD7 and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 17.

In some embodiments, the at least one modulatory domain is CD30L and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 18.

In some embodiments, the at least one modulatory domain is CD40 and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 19.

In some embodiments, the at least one modulatory domain is CD70 and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 20.

In some embodiments, the at least one modulatory domain is CD83 and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 21.

In some embodiments, the at least one modulatory domain is HLA-G and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 22.

In some embodiments, the at least one modulatory domain is MICA and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 23.

In some embodiments, the at least one modulatory domain is MICB and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 24.

In some embodiments, the at least one modulatory domain is HVEM and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 25.

In some embodiments, the at least one modulatory domain is lymphotoxin beta receptor and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 26.

In some embodiments, the at least one modulatory domain is 3/TR6 and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 27.

In some embodiments, the at least one modulatory domain is ILT3 and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 28.

In some embodiments, the at least one modulatory domain is ILT4 and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 29.

In some embodiments, the at least one modulatory domain is CTL4 and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 30.

In some embodiments, the at least one modulatory domain is Tim3 and comprises an amino acid sequence having at least about 70%, at least about 75%, at least about 80%, at least about 85%, at least about 90%, at least about 95%, at least about 98%, at least about 99%, or 100% amino acid sequence identity to SEQ ID NO: 31.

In some aspects, the disclosed fusion polypeptides do not comprise a major histocompatibility complex (“MHC”) polypeptide sequence, variant, or fragment thereof.

In some embodiments, the fusion polypeptide comprises a targeting moiety, wherein the targeting moiety comprises an scFv domain that targets a corresponding myc tag, and wherein the fusion polypeptide comprises no MHC polypeptide sequence, variant, or fragment thereof.

In some embodiments, the fusion polypeptide comprises a targeting moiety, wherein the targeting moiety comprises an scFv domain that targets a corresponding myc tag and a modulatory domain comprising an CD28-antibody or antibody fragment thereof, and wherein the fusion polypeptide comprises no MHC polypeptide sequence, variant, or fragment thereof.

In some embodiments, the fusion polypeptide comprises a targeting moiety, wherein the targeting moiety comprises an scFv domain that targets a corresponding myc epitope and an modulatory domain comprising a 4-1BBL polypeptide, and wherein the fusion polypeptide comprises no MHC polypeptide sequence, variant, or fragment thereof.

In some embodiments, the fusion polypeptide is a universal fusion polypeptide. In some embodiments, the fusion polypeptide is a split, universal, and programmable (“SUPRA”) fusion polypeptide (e.g., Cho J et al. (2018) Cell 173, 1426-1438). In some embodiments, the fusion polypeptide is a biotin-binding immune receptor (“BBIR”) fusion polypeptide (e.g., Urbanska K et al. (2012) Cancer Res. 72(7): 1844-1852).

### Nucleic Acids

In some aspects, the present disclosure provides nucleic acids comprising a nucleotide sequence encoding a fusion polypeptide, wherein the fusion polypeptide comprises a targeting domain, and wherein the targeting domain comprises a single chain variable (scFv) domain. In some aspects, the present disclosure provides nucleic acids comprising a nucleotide sequence encoding a fusion polypeptide, wherein the fusion polypeptide comprises a targeting domain comprising an scFv domain, and wherein the fusion polypeptide comprises no MHC polypeptide sequence, variant, or fragment thereof.

In some embodiments, the nucleic acids comprise nucleic acid sequences encoding 1 scFv domain. In other embodiments, the nucleic acids comprise nucleic acid sequences encoding at least 2, at least 3, at least 4, at least 5, at least 6, at least 7, at least 8, at least 9, at least 10, or more scFv domains.

The targeting moiety encoded by the nucleic acids can target any proteinaceous or non-proteinaceous sequence that is selectively presented on the CAR-T cell, for example at least one targeted moiety. The targeting moiety encoded by the nucleic acids function to recognize and to selectively modulate a CAR-T cell. Non-limiting examples of suitable targets for the targeting moiety include a myc epitope, a FLAG epitope, a His epitope, a HA epitope, a GST epitope, a TAP epitope, a V5 epitope, CBP epitope, a MBP epitope, or a fluorescent epitope.

In some embodiments, the scFv domains encoded by the nucleic acids comprise 1 targeting moiety. In other embodiments, the scFv domains encoded by the nucleic acids comprise at least 2, 3, 4, 5, 6, 7, 8, 9, 10, or more targeting moieties. In some embodiments, the scFv domains encoded by the nucleic acids comprise less than 10, less than 9, less than 8, less than 7, less than 6, less than 5, less than 4, less than 3, or less than 2 targeting moieties.

In some embodiments, the nucleic acids provided herein encode an antibody or a fragment or derivative thereof comprising a targeting moiety. Non-limiting examples of suitable antibodies, fragments, or derivatives thereof include myc antibodies, fragments, or derivatives thereof; FLAG antibodies, fragments, or derivatives thereof; His antibodies, fragments, or derivatives thereof; HA antibodies, fragments, or derivatives thereof; GST antibodies, fragments, or derivatives thereof; TAP antibodies, fragments, or derivatives thereof; V5 antibodies, fragments, or derivatives thereof; CBP antibodies, fragments, or derivatives thereof; MBP antibodies, fragments, or derivatives thereof; or fluorescent antibodies, fragments, or derivatives thereof. Myc antibodies, fragments, or derivatives thereof include, for example, 9E10 clone, 9E10.3 clone, 27H46L35 clone, and 9E11 clone. FLAG antibodies, fragments, or derivatives thereof include, for example, 20H18L16, 20H1L23, 8H2L5, 8H8L17, 8HCLC, L5, and FG4R. HA antibodies, fragments, or derivatives thereof include, for example, HA.C5, 4C12, 16B12, and HA-7. His antibodies, fragments, or derivatives thereof include, for example, 4E3D10H2/E3, HIS.H8, 4A12E4, and 21HCLC. In other embodiments, the antibodies, fragments, or derivatives thereof can include tags including, for example, horseradish peroxidase (“HRP”), biotin, a fluorescent tag, FITC, or phycoerythrin. In some embodiments, the antibodies, fragments, or derivatives thereof is a monoclonal. In other embodiments, the antibodies, fragments, or derivatives thereof is a polyclonal. In yet other embodiments, the antibodies, fragments, or derivatives thereof is an oligoclonal.

The targeting moiety of the fusion polypeptide encoded by the nucleic acid can be located at any position within the scFv domain. In some embodiments, targeting moiety is located at an N-terminal end of the polypeptide. In certain embodiments, the fusion polypeptide may comprise a plurality of N-termini and a plurality of targeting moieties. In other embodiments, targeting moiety is located at a C-terminal end of the polypeptide. In certain embodiments, the polypeptide may comprise a plurality of C-termini and a plurality of targeting moieties. In yet other embodiments, the targeting moiety is located in the middle or substantially the middle of the polypeptide or in other extracellular domains of the polypeptide.

In some embodiments, the nucleic acid further comprises a nucleic acid encoding at least 1 modulatory domain. The modulatory domains of the present disclosure function as a costimulatory signal, a cytokine, and/or an inhibitory signal to modulate CAR-T cells. In some embodiments, the nucleic acid further encodes at least 2, at least 3, at least 4, at least 5, at least 6, at least 7, at least 9, at least 10, or more modulatory domains. In some embodiments, the nucleic acid encodes less than 10, less than 9, less than 8, less than 7, less than 6, less than 5, less than 4, less than 3, or less than 2 modulatory domains.

In some embodiments, the at least one modulatory domain encoded by the nucleic acid is selected from an anti-CD28 polypeptide, a 4-1BBL polypeptide, a GITR polypeptide, CD27 polypeptide, a B7-1 polypeptide, a B7-2 polypeptide, an ICOS-L polypeptide, an OX-40L polypeptide, a CD80 polypeptide, a CD86 polypeptide, a PD-L1 polypeptide, a FasL polypeptide, and a PD-L2 polypeptide. In some embodiments, the at least one modulatory domain is selected from CD7, CD30L, CD40, CD70, CD83, HLA-G, MICA, MICB, HVEM, lymphotoxin beta receptor, 3/TR6, ILT3, CTLA4, PD1, PDL1, and ILT4.

### CAR-Expressing Cells

In some aspects, provided herein are CAR-expressing T cells (CAR-T cells). CAR-T cells are T cells that have been genetically engineered to produce an artificial T-cell receptor. In some embodiments the CAR-expressing cell may be a CAR-expressing peripheral blood mononuclear (PBMC) cell, a CAR-expressing natural killer (NK) NK cell, or a CAR-expressing CD8 cell.

In some embodiments, the CAR-expressing cell comprises a targeted moiety that is recognized by a targeting moiety of the fusion polypeptide. Non-limiting examples of suitable targeted moieties include a myc epitope, a FLAG epitope, a His epitope, a HA epitope, a GST epitope, a TAP epitope, a V5 epitope, a CBP epitope, an MBP epitope, or a fluorescent epitope.

In some embodiments, the CAR-expressing cell is an autologous CAR-expressing cell. In other embodiments, the CAR-expressing cell is an allogenic CAR-expressing cell.

In some embodiments, the CAR-expressing cell is a commercially available and/or approved CAR-expressing cell that has been reengineered to include an epitope, for example a myc epitope, into the CAR. Examples commercially available and/or approved CAR-T cell therapies are Kymriah® (tisagenlecleucel) and Yescarta® (axicabtagene ciloleucel).

In some embodiments, the CAR-expressing cell is a switchable CAR-expressing cell (e.g., Raj D, et al., Gut 2018; 0:1-13). In other embodiments, the CAR-expressing cell is a fluorescent CAR-expressing cell, for example, an anti-FITC CAR-T cell (e.g., Tamada K et al., Clin Cancer Res (2012) 18(23): 6436-45). In yet other embodiments, the CAR-expressing cell is a peptide-specific switchable CAR-expressing cell (sCAR-T) (e.g., Rodgers D et al., Proc Natl Acad Sci USA (2016) E459-E468). In some embodiments, the CAR-expressing cell is a universal CAR-T cell. In some embodiments, the CAR-expressing cell expresses an scFv that is specific for an antigen that is present on the surface of a tumor cell or an infected cell. In some embodiments, the scFv is specific for CD19, CD22, gp120, ROR1, or FGFR4.

### Compositions

In some aspects, the present disclosure provides compositions comprising a fusion polypeptide or a nucleic acid according to an embodiment disclosed and described herein. In some embodiments, the compositions further comprise a CAR-expressing cell according to an embodiment disclosed and described herein, for example, wherein the CAR-expressing cell comprises a targeted moiety that is recognized by the targeting moiety of the fusion polypeptide.

In some embodiments, the composition further comprises a pharmaceutically acceptable excipient. Non-limiting examples of pharmaceutically acceptable excipients include, for example, those described in “Remington: The Science and Practice of Pharmacy”, 19th Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc. In some embodiments, the composition is suitable for administration to a subject, for example, a sterile composition. In some embodiments, the composition is suitable for administration to a human subject, for example, the composition is sterile and is free of detectable pyrogens and/or other toxins.

In some embodiments, the composition comprises other components, such as pharmaceutical grades of mannitol, lactose, starch, magnesium stearate, sodium saccharin, talcum, cellulose, glucose, sucrose, magnesium, carbonate, and the like. In some embodiments, the compositions comprise a pharmaceutically acceptable auxiliary substance as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

In some embodiments, the compositions are in an aqueous solution, powder form, granules, tablets, pills, suppositories, capsules, suspensions, sprays, and the like. The composition may comprise a pharmaceutically acceptable excipient, a pharmaceutically acceptable salt, diluents, carriers, vehicles and such other inactive agents well known to the skilled artisan. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions may be prepared using conventional techniques that may include sterile isotonic saline, water, 1,3-butanediol, ethanol, 1,2-propylene glycol, polyglycols mixed with water, Ringer's solution, etc. In one aspect, a coloring agent is added to facilitate in locating and properly placing the composition to the intended treatment site.

Compositions may include a preservative and/or a stabilizer. Non-limiting examples of preservatives include methyl-, ethyl-, propyl-parabens, sodium benzoate, benzoic acid, sorbic acid, potassium sorbate, propionic acid, benzalkonium chloride, benzyl alcohol, thimerosal, phenylmercurate salts, chlorhexidine, phenol, 3-cresol, quaternary ammonium compounds (QACs), chlorbutanol, 2-ethoxyethanol, and imidurea.

To control tonicity, the composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8, e.g., between 6.5 and 7.5, or between 7.0 and 7.8.

The composition can be administered by any appropriate route, which will be apparent to the skilled person depending on the disease or condition to be treated. Typical routes of administration include intravenous, intra-arterial, intramuscular, subcutaneous, intracranial, intranasal or intraperitoneal.

In some embodiments, the composition may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

The composition can comprise a pharmaceutically acceptable excipient, a pharmaceutically acceptable salt, diluents, carriers, vehicles and such other inactive agents well known to the skilled artisan. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions may be prepared using conventional techniques that may include sterile isotonic saline, water, 1,3-butanediol, ethanol, 1,2-propylene glycol, polyglycols mixed with water, Ringer's solution, etc. In one aspect, a coloring agent is added to facilitate in locating and properly placing the composition to the intended treatment site.

In one aspect is provided a kit of parts comprising the above-mentioned composition (fusion polypeptide, nucleic acid and/or at least one CAR-expressing cell). The kit may further comprise a document or an instruction manual that describes a protocol for preparing the fusion polypeptides or nucleic acids, culturing and/or preparing the CAR-expressing cells, and/or administering to a subject in need thereof.

### Methods of Use and Treatment

Provided herein are methods of use and treatment of a subject in need thereof. In some embodiments, provided herein are fusion polypeptides and/or nucleic acids for use in therapy. In other embodiments, provided herein are fusion polypeptides and/or nucleic acids for use in the production of a medicament.

Also provided herein are in vivo methods of modulating a CAR-expressing cell, comprising administering to a subject the subject a fusion polypeptide, a nucleic acid, or a composition according to an embodiment disclosed and described herein, wherein the CAR-expressing cell comprises an targeted moiety that is recognized by the targeting moiety of the fusion polypeptide.

In some embodiments, modulating the CAR-expressing cell induces proliferation of the CAR-expressing cell, induces differentiation of the CAR-expressing cell, stimulates the CAR-expressing cell to induce cytotoxic effects, inhibits the CAR-expressing cell, induces apoptosis or necrosis in the CAR-expressing cell, induces quiescence, or any combination thereof.

Also provided herein are methods of treating a subject, comprising administering to the subject an amount of a fusion polypeptide, a nucleic acid, or a composition according to an embodiment disclosed and described herein. In some embodiments, the subject is further administered a CAR-expressing cell, wherein the CAR-expressing cell comprises a targeted moiety that is recognized by the targeting moiety of the fusion polypeptide.

In some embodiments, the CAR-expressing cell is administered prior to, subsequent to, or concurrently with the fusion polypeptide.

In some embodiments, the subject has a disease associated with expression of a tumor antigen or a disease associated with a pathogen. In some embodiments, the disease associated with expression of a tumor antigen is a proliferative disease, a precancerous condition, a cancer, or a non-cancer related indication associated with expression of the tumor antigen.

In some embodiments, the cancer is chronic lymphocytic leukemia (CLL), acute leukemia, acute lymphoid leukemia (ALL), B-cell acute lymphoid leukemia (B-ALL), T-cell acute lymphoid leukemia (T-ALL), chronic myelogenous leukemia (CML), B-cell prolymphocytic leukemia, blastic plasmacytoid dendritic cell neoplasm, Burkitt's lymphoma, diffuse large B-cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia.

In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (“CNS”), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of the cancers, and metastatic lesions of the cancers. In some embodiments, the cancer is leukemia or lymphoma, for example, lymphoblastic lymphoma or B-cell Non-Hodgkin's lymphoma

In some embodiments, the pathogen is a viral pathogen. Non-limiting examples of viruses amenable to the present disclosure include human immunodeficiency viruses (e.g., HIV-1 and HIV-2), influenza viruses (e.g., influenza A, B and C viruses), papillomaviruses, coronaviruses (e.g., human respiratory coronavirus), hepatitis viruses (e.g., hepatitis viruses A to G), or herpesviruses (e.g., HSV 1-9). In some embodiments, the viral pathogen is HIV.

In some embodiments, modulation of the CAR-expressing cell induces proliferation of the CAR-expressing cell, induces differentiation of the CAR-T cell, stimulates the CAR-expressing cell, inhibits the CAR-expressing cell, or any combination thereof.

In some embodiments, the modulation of the CAR-expressing cell is via direct targeting of the CAR-expressing cell using a fusion polypeptide comprising a targeting moiety to bind to a targeted moiety incorporated in the CAR sequence to either activate the CAR-expressing cell and specifically deliver a defined costimulatory signal or specifically provide an inhibitory sequence.

In some embodiments, the modulation of the CAR-expressing cell is at least about 0.01-fold higher, at least about 0.1-fold higher, at least about 0.5-fold higher, at least about 1.0-fold higher, at least about 1.5-fold higher, at least about 2-fold higher, at least about 2.5-fold higher, at least about 3-fold higher, at least about 4-fold higher, at least about 5-fold, about at least 10-fold higher, at least about 15-fold higher, at least about 20-fold higher, at least about-25 fold higher, or more as compared to modulation of a CAR-expressing cell without the targeted moiety, e.g., domain or epitope, that is recognized by the targeting moiety of the fusion polypeptide. In some embodiments, the modulation of the CAR-expressing cell is about 0.01-fold higher, about 0.1-fold higher, about 0.5-fold higher, about 1.0-fold higher, about 1.5-fold higher, about 2-fold higher, about 2.5-fold higher, about 3-fold higher, about 4-fold higher, about 5-fold, about 10-fold higher, about 15-fold higher, about 20-fold higher, about-25 fold higher, or more as compared to modulation of a CAR-expressing cell without the targeted moiety that is recognized by the targeting moiety of the fusion polypeptide.

In some embodiments, the subject experiences a reduction or elimination in an inflammatory response, a cytokine storm, or at least one off-target effect, or any combination thereof as compared to a control subject. In some embodiments, the subject experiences a reduction in a symptom or a side effect as compared to a control subject. In some embodiments, the symptom is cytokine-release syndrome (CRS), a neurologic toxicity, B-cell aplasia, tumor lysis syndrome (TLS), anaphylaxis, fever, joint/muscle aches, shortness of breath, low blood pressure, confusion, or a seizure. The control subject can be a subject who receives a placebo control, a CAR-expressing cell without the epitope that is recognized by the targeting domain of the fusion polypeptide, or a fusion polypeptide having a targeting domain that is not recognized by the CAR-expressing cell and/or the epitope of the CAR-expressing cell. For example, a subject could be a subject who receives: (1) axicabtagene ciloleucel, a CAR-T cell therapy, modified to express a myc epitope and (2) a fusion polypeptide comprising a single chain variable (scFv) domain having a myc targeting domain and a PD-L1 inhibitory polypeptide wherein the fusion polypeptide comprises no major histocompatibility complex (MHC) polypeptide sequence, variant, or fragment thereof and the control subject could be a subject who receives: (1) axicabtagene ciloleucel, CAR-T cell therapy, that does not express a myc epitope and (2) a fusion polypeptide comprising a single chain variable (scFv) domain having a targeting moiety specific for a myc epitope and a PD-L1 inhibitory polypeptide wherein the fusion polypeptide comprises no major histocompatibility complex (“MHC”) polypeptide sequence, variant, or fragment thereof.

The foregoing list of modifications is intended to be exemplary in nature, and the skilled artisan will appreciate that other modifications may be possible or desirable in certain applications. For brevity, therefore, certain systems, methods and compositions of the present disclosure are exemplified by reference to particular fusion polypeptides, but it should be understood that the fusion polypeptides may be modified or used in modified ways that do not alter the exemplified operating principles. Such modifications are within the scope of the present disclosure.

### EXAMPLES

The following Examples are illustrative and are not intended to limit the scope or content of the invention in any way.

### Example 1: Construction of Modulatory Fusion Polypeptides Targeting an HIV-Specific CAR-T Cell Construct Incorporating an Extracellular Myc Epitope as a Targeted Moiety

It was hypothesized that the mechanisms underlying mobilization of the T cell response could be exploited to develop biologics to selectively activate, expand and/or differentiate CAR-T cells targeting malignant or infected cells.

It is understood that three signals usually provided by antigen presenting cells (“APC”) activate and expand antigen-specific T cells capable of potently eliminating malignant or infected cells: 1) an antigen-specific activation signal provided by TCR recognition of its cognate peptide presented by an MHC Class I molecule; 2) a costimulatory signal provided by one of several possible costimulatory molecules (e.g., CD28, 4-1BB, GITR) to fully activate the T cell or PD-1 to suppress the T cells; and 3) a proliferative and/or differentiation signal from one of several possible cytokines. The crucial role of the costimulatory signal is underscored by the state of non-responsiveness induced if the primary TCR signal is delivered without the appropriate costimulatory signals.

Using sophisticated protein design and engineering principles novel biological molecules were constructed having highly stable dimeric IgG Fc-fusion polypeptides, which mimic APC functions that specifically bind to the CAR to provide the primary activation signal and also target the CAR-T cell for the delivery of defined costimulatory, coinhibitory or cytokine signals. Based on extensive preliminary studies evaluating multiple linkage and spacer strategies, the optimal scaffold that forms stable assemblies to efficiently bind to the CAR and deliver the CAR-specific and costimulatory signals required to activate, expand and differentiate CAR-T cells was identified (FIG. 1).

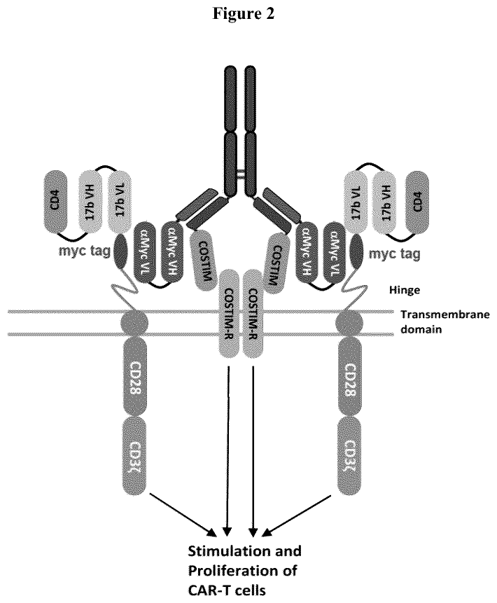

As an initial proof of concept demonstration of the fusion peptides, native IgG1 Fc protein was used as the scaffold to construct the modulatory fusion polypeptides. This Fc protein is a homodimer of two identical immunoglobulin Fc CH2-CH3 domains stabilized through two disulfide bonds linking the CH2 domains. Each Fc domain was linked to a single chain variable (“scFv”) derived from the 9E10 antibody targeting a myc epitope (the targeting moiety) which can be generated and incorporated in a CAR construct. This scFv was fused to a domain of T cell modulatory function and presented in the context of an Fc fusion polypeptides. These fusion polypeptides have been termed a CAR-T-cell activator (“CAR-Tac”). The interaction of the CAR-Tac with a HIV gp120-specific CAR containing an incorporated myc epitope (the targeted moiety) is illustrated in FIG. 2.

Importantly, this strategy is applicable irrespective of the molecule that the CAR-T cell recognizes in the targeted cell population. While prior efforts have focused on the control of target cell recognition, the “re-directing” of CAR-T cells to target cell populations and on the introduction of binary/logic-gate-based mechanisms to control T cell activity, it is believed that this is the first strategy to specifically and directly modulate the in vivo expansion and differentiation of CAR-T cells via direct targeting of the CAR-T cells themselves.

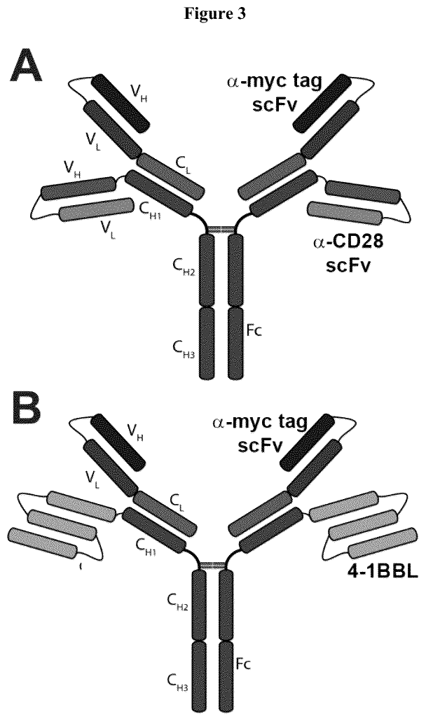

Fusion polypeptides possessing a first scFv domain (the targeting domain) specific to myc to target myc-tag expressing CAR-T cells and a second scFv domain linked to an anti-CD28 polypeptide, which stimulates the B7-CD28 costimulatory pathway (the modulatory domain) (FIG. 3A) or a single chain trimeric 4-1BBL costimulatory ligand to active the 4-1BB costimulatory pathway (FIG. 3B) were generated to activate the CD28 or 4-1BBL costimulatory pathways, respectively. It is contemplated that ligands for other costimulatory pathways such as OX-40, GITRL, ICOS or CD27 pathways or cytokines such as IL-2, IL-12 or IL-15 or ligands, scFvs and antibodies that bind cytokine receptors could be incorporated into the CAR-Tac to provide a potent costimulation and/or cytokine signal to fully activate, expand and differentiate the CAR-T cells or alternatively a PD1-L molecule to bind to the inhibitory receptor PD-1 to suppress the CAR-T cell if it is deleterious to the subject through triggering a severe inflammatory response such as cytokine storm or destructive off-target effect.

### Example 2: Construction of an HIV-Specific CAR-T Cell Construct Possessing an Extracellular Myc Epitope as a Targeted Moiety

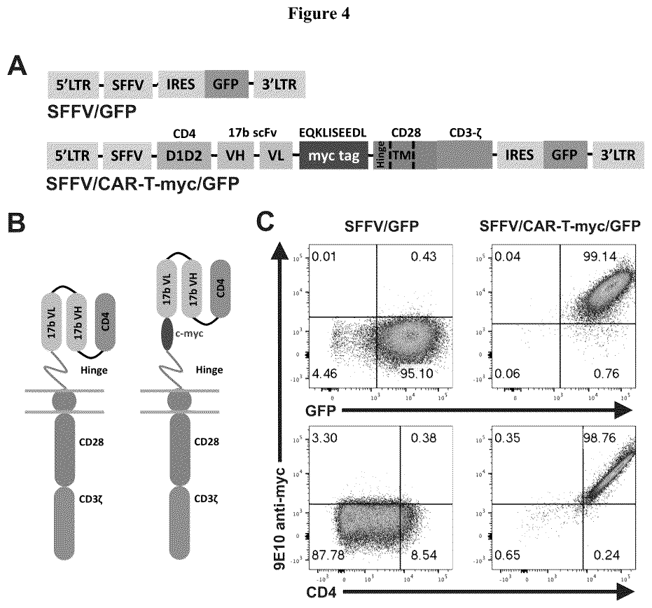

To demonstrate that this strategy could be used to stimulate CAR-T cells, a construct encoding an HIV-specific CAR-T molecule (FIG. 4A, left panel), which confers potent anti-HIV in vitro activity to transduced CD8⁺ T cells was obtained (provided by Dr. Ed Berger National Institute of Allergy and Infectious Disease (NIAID) and the National Institutes of Health (NIH)). The bispecific CD4-10-17b CAR-T cell construct (anti-HIV CAR-T) encodes the gp120 binding region of human CD4 (D1D2) coupled to a scFv derived from human mAb 17b specific for the HIV co-receptor site. A 10-aa myc epitope (SEQ ID NO: 1) derived from the human c-myc protein was incorporated between the 17b scFv and the CD28 transmembrane domain (FIGS. 4A and 3B, right panel) of the CAR construct. To facilitate expression of CAR-T-myc in primary cells, a SFFV-regulated lentiviral vector, SFFV-GFP, which encodes a GFP reporter gene that is expressed well in T cells was used (FIG. 4A, top panel) to produce a HIV-specific CAR-T cell construct that includes the myc epitope (FIG. 4B, lower panel). Together this data demonstrated that the myc epitope is accessible in this location for binding to the α-myc epitope antibody (FIG. 4C).

Furthermore, as a “self”-protein-derived peptide, this epitope is anticipated to have low to no immunogenicity in humans. Expression of the myc-CAR construct was evaluated by flow cytometric analysis of CD8⁺ Jurkat/MA T cells, which do not express CD4 or an endogenous surface TCR, after transduction with the SFFV/CAR-T-myc/GFP lentivirus. Transduction of the Jurkat/MA cells with the SFFV/GFP (empty control vector) or SFFV/CAR-T-myc/GFP lentivirus is indicated by expression of the GFP reporter gene and expression of the CAR by demonstration of CD4 expression (FIG. 4C). Jurkat/MA cells transduced with the SFFV/CAR-T-myc/GFP lentivirus express the CAR as indicated by staining for CD4 (FIG. 4C, lower right panel) which is also detectable by binding of the anti-myc epitope antibody, 9E10 (FIG. 3C, lower right panel). This data indicated successful construction of a CAR possessing a myc epitope that is accessible to the 9E10 anti-myc scFv incorporated in the modulatory fusion polypeptide.

### Example 3: Stimulation of Primary T Cells by the Modulatory Fusion Polypeptide

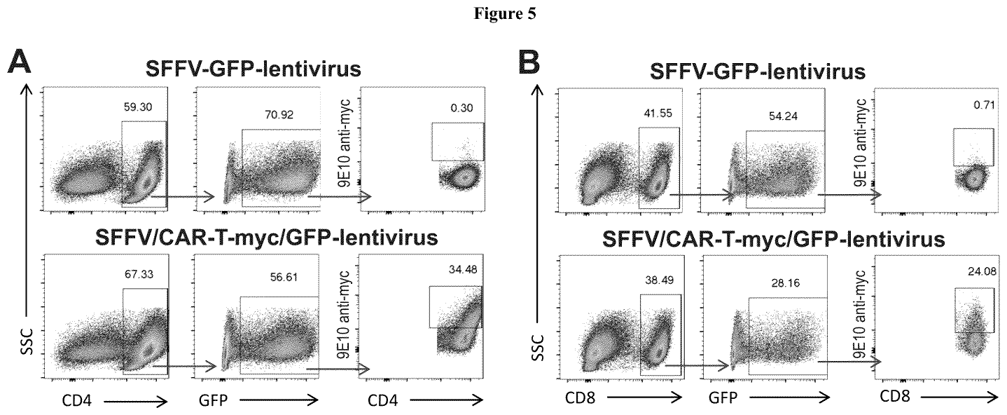

Purified human CD3⁺ T cells were transduced with the control SFFV-GFP-lentivirus or the SFFV/CAR-T-myc/GFP lentivirus; transduction was scored by GFP expression, and accessibility of the myc epitope was indicated by binding to antibody 9E10. As shown in FIG. 5, the 9E10 anti-myc epitope antibody bound to ˜35% of the transduced CD4⁺ T cells and ˜25% of the transduced CD8⁺ T cells as compared to <1% for T cells transduced with the control lentivirus.

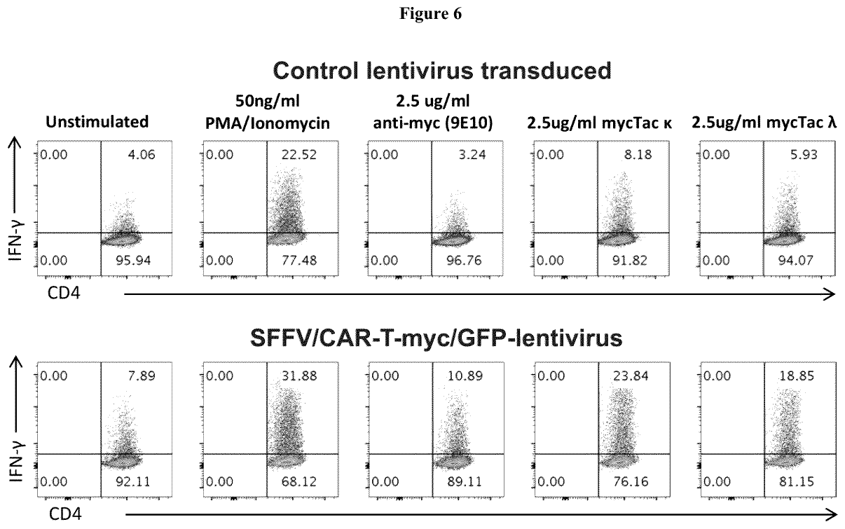

To determine the capacity of the modulatory fusion polypeptides (i.e., the CAR-Tacs) to specifically activate the myc epitope-expressing CAR-T cells, T cells transduced with a control SFFV-GFP-lentivirus or T cells transduced with the SFFV/CAR-T-myc/GFP lentivirus were stimulated as indicated (FIG. 6). After overnight incubation, T cell activation in CD4+ CAR-T cells was determined by flow cytometric quantification of IFN-γ expression after gating the cells for expression of the lentivector GFP marker gene and CD4. As shown in FIG. 6, stimulation of T cells transduced with the control SFFV-GFP-lentivirus or with the SFFV/CAR-T-myc/GFP lentivirus with PMA/ionomycin increased IFN-γ expression by the CAR-expressing CD4+ T cells by about 4- to 5-fold, as compared to unstimulated cells. While treatment with the 9E10 anti-myc epitope antibody did not increase IFN-γ production by the T cells transduced with the control SFFV-GFP-lentivirus, it increased IFN-γ production by the myc epitope-expressing CAR-T cells transduced with the SFFV/CAR-T-myc/GFP lentivirus by almost 3-fold.

The CD4⁺ T cells were also stimulated with CAR-Tac/α-CD28 with the α-CD28 domain linked to the C_{L} domain either using a kappa or a lambda light chain as illustrated in FIG. 3A. Both modulatory fusion polypeptides (i.e., CAR-Tacs) stimulated ˜2-fold higher IFN-γ production by the CAR-T cells transduced with the SFFV/CAR-T-myc/GFP lentivirus than an equivalent concentration of the anti-myc antibody, with the kappa-linked CAR-Tac/α-CD28 inducing greater than a 2-fold higher IFN-γ production, indicating the additional activation conferred by the α-CD28 domain, with minimal stimulation of the CD4⁺ T cells transduced with a control SFFV-GFP-lentivirus (FIG. 6). This indicates that the modulatory fusion polypeptide linkage, which enables cross-linking of adjacent CAR and co-stimulatory molecules and formation of the immunological synapse, greatly augments its capacity to activate CAR-T cells.

It is contemplated that the kappa chain linkage of the α-CD28 domain in the modulatory fusion polypeptide may enable a more potent signal than the lambda chain linkage.

### INCORPORATION BY REFERENCE

All references mentioned herein are hereby incorporated by reference in their entirety as if each individual reference was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES

1. Park J H, Geyer M B, Brentjens R J. CD19-targeted CAR T-cell therapeutics for hematologic malignancies: interpreting clinical outcomes to date. Blood. 2016; 127(26):3312-20
2. Zhang W-y, Wang Y, Guo Y-I, Dai H-r, Yang Q-m, Zhang Y-j, et al. Treatment of CD20-directed Chimeric Antigen Receptor-modified T cells in patients with relapsed or refractory B-cell non-Hodgkin lymphoma: an early phase IIa trial report. Signal Transduction And Targeted Therapy. 2016; 1:16002
3. Fry T J, Shah N N, Orentas R J, Stetler-Stevenson M, Yuan C M, Ramakrishna S, et al. CD22-targeted CART cells induce remission in B-ALL that is naive or resistant to CD19-targeted CAR immunotherapy. 2018; 24(1):20-8
4. June C H, O'Connor R S, Kawalekar O U, Ghassemi S, Milone M C. CART cell immunotherapy for human cancer. Science. 2018; 359(6382):1361-5. Epub 2018/03/24
5. Scholler J, Brady T L, Binder-Scholl G, Hwang W T, Plesa G, Hege K M, et al. Decade-long safety and function of retroviral-modified chimeric antigen receptor T cells. Science translational medicine. 2012; 4(132): 132ra53
6. Deeks S G, Wagner B, Anton P A, Mitsuyasu R T, Scadden D T, Huang C, et al. A phase II randomized study of HIV-specific T-cell gene therapy in subjects with undetectable plasma viremia on combination antiretroviral therapy. Molecular therapy: the journal of the American Society of Gene Therapy. 2002; 5(6):788-97. Epub 2002/05/25
7. Mitsuyasu R T, Anton P A, Deeks S G, Scadden D T, Connick E, Downs M T, et al. Prolonged survival and tissue trafficking following adoptive transfer of CD4zeta gene-modified autologous CD4(+) and CD8(+) T cells in human immunodeficiency virus-infected subjects. Blood. 2000; 96(3):785-93. Epub 2000/07/27
8. Levine B L, Humeau L M, Boyer J, MacGregor R-R, Rebello T, Lu X, et al. Gene transfer in humans using a conditionally replicating lentiviral vector. Proceedings of the National Academy of Sciences. 2006; 103(46):17372-7
9. Liu L, Patel B, Ghanem M H, Bundoc V, Zheng Z, Morgan R A, et al. Novel CD4-Based Bispecific Chimeric Antigen Receptor Designed for Enhanced Anti-HIV Potency and Absence of HIV Entry Receptor Activity. Journal of virology. 2015; 89(13):6685-94
10. Ghanem M R, Bolivar-Wagers S, Dey B, Hajduczki A, Vargas-Inchaustegui D A, Danielson D T, et al. Bispecific chimeric antigen receptors targeting the CD4 binding site and high-mannose Glycans of gp120 optimized for anti-human immunodeficiency virus potency and breadth with minimal immunogenicity. Cytotherapy. 2018. Epub 2018/01/08
11. Ali A, Kitchen S G, Chen I S, Ng H L, Zack J A, Yang 00. HIV-1-Specific Chimeric Antigen Receptors Based on Broadly Neutralizing Antibodies. Journal of virology. 2016; 90(15):6999-7006. Epub 2016/05/27
12. Hale M, Mesojednik T, Romano Ibarra G S, Sahni J, Bernard A, Sommer K, et al. Engineering HIV-Resistant, Anti-HIV Chimeric Antigen Receptor T Cells. Molecular Therapy. 2017; 25(3):570-9
13. Liu B, Zou F, Lu L, Chen C, He D, Zhang X, et al. Chimeric Antigen Receptor T Cells Guided by the Single-Chain Fv of a Broadly Neutralizing Antibody Specifically and Effectively Eradicate Virus Reactivated from Latency in CD4+ T Lymphocytes Isolated from HIV-1-Infected Individuals Receiving Suppressive Combined Antiretroviral Therapy. Journal of virology. 2016; 90(21):9712-24. Epub 2016/08/19
14. Zhen A, Peterson C W, Carrillo M A, Reddy S S, Youn C S, Lam B B, et al. Long-term persistence and function of hematopoietic stem cell-derived chimeric antigen receptor T cells in a nonhuman primate model of HIV/AIDS. PLoS pathogens. 2017; 13(12):e1006753
15. Hale M, Mesojednik T, Romano Ibarra G S, Sahni J, Bernard A, Sommer K, et al. Engineering HIV-Resistant, Anti-HIV Chimeric Antigen Receptor T Cells. Mol Ther. 2017; 25(3):570-9. Epub 2017/02/02
16. Tang Q, Smith J A, Szot G L, Zhou P, Alegre M L, Henriksen K J, et al. CD28/B7 regulation of anti-CD3-mediated immunosuppression in vivo. J Immunol. 2003; 170(3):1510-6. Epub 2003/01/23
17. Grosse-Hovest L, Hartlapp I, Marwan W, Brem G, Rammensee H G, Jung G. A recombinant bispecific single-chain antibody induces targeted, supra-agonistic CD28-stimulation and tumor cell killing. European journal of immunology. 2003; 33(5):1334-40. Epub 2003/05/06
18. Bartkowiak T, Singh S, Yang G, Galvan G, Haria D, Ai M, et al. Unique potential of 4-1BB agonist antibody to promote durable regression of HPV+ tumors when combined with an E6/E7 peptide vaccine. Proceedings of the National Academy of Sciences of the United States of America. 2015; 112(38):E5290-9. Epub 2015/09/10
19. Bartkowiak T, Curran M A. 4-1BB Agonists: Multi-Potent Potentiators of Tumor Immunity. Frontiers in oncology. 2015; 5: 117. Epub 2015/06/25
20. Shuford W W, Klussman K, Tritchler D D, Loo D T, Chalupny J, Siadak A W, et al. 4-1BB costimulatory signals preferentially induce CD8+ T cell proliferation and lead to the amplification in vivo of cytotoxic T cell responses. The Journal of experimental medicine. 1997; 186(1):47-55. Epub 1997/07/07
21. Joseph A, Zheng J H, Follenzi A, Dilorenzo T, Sango K, Hyman J, et al. Lentiviral vectors encoding human immunodeficiency virus type 1 (HIV-1)-specific T-cell receptor genes efficiently convert peripheral blood CD8 T lymphocytes into cytotoxic T lymphocytes with potent in vitro and in vivo HIV-1-specific inhibitory activity. Journal of virology. 2008; 82(6):3078-89. Epub 2008/01/11
22. Zuccolotto G, Fracasso G, Merlo A, Montagner I M, Rondina M, Bobisse S, et al. PSMA-specific CAR-engineered T cells eradicate disseminated prostate cancer in preclinical models. PloS one. 2014; 9(10):e109427. Epub 2014/10/04
23. Kieback E, Charo J, Sommermeyer D, Blankenstein T, Uckert W. A safeguard eliminates T cell receptor gene-modified autoreactive T cells after adoptive transfer. Proceedings of the National Academy of Sciences of the United States of America. 2008; 105(2):623-8. Epub 2008/01/10
24. Flerin N C, Chen H, Glover T D, Lamothe P A, Zheng J H, Fang J W, et al. T-Cell Receptor (TCR) Clonotype-Specific Differences in Inhibitory Activity of HIV-1 Cytotoxic T-Cell Clones Is Not Mediated by TCR Alone. Journal of virology. 2017; 91(6). Epub 2017/01/13

TABLE 2
Polypeptide Sequences

| SEQ ID NO. | Name | Polypeptide sequence | Reference/notes |
|---|---|---|---|
| SEQ ID NO: 1 | myc | EQKLISEEDL | |
| SEQ ID NO: 2 | FLAG | DYKDDDK | |
| SEQ ID NO: 3 | His5 | HHHHH | |
| SEQ ID NO: 4 | His6 | HHHHHH | |
| SEQ ID NO: 5 | Hemagglutinin | YPYDVPDYA | |
| | (HA) | | |
| SEQ ID NO: 6 | Bispecific | DIELTQSPASLAVSLGQRATISCRASESVEYYVTSLMQWYQQKPGQPPKLLIFAASNVESGVPARFSG | PMID 12731059 |
| | anti-CD28 | SGSGTNFSLNIHPVDEDDVAMYFCQQSRKVPYTFGGGTKLEIKRGGGGSGGGGSGGGGSQVKLQQS | |
| | scFv | GPGLVTPSQSLSITCTVSGFSLSDYGVHWVRQSPGQGLEWLGVIWAGGGTNYNSALMSRKSISKDN | |
| | | SKSQVFLKMNSLQADDTAVYYCARDKGYSYYYSMDYWGQGTTVTVSS | |
| SEQ ID NO: 7 | 41BBL single | DPAGLLDLRQGMFAQLVAQNVLLIDGPLSWYSDPGLAGVSLTGGLSYKEDTKELVVAKAGVYYVF | (G4S)7 linker 1, |
| | chain trimer | FQLELRRVVAGEGSGSVSLALHLQPLRSAAGAAALALTVDLPPASSEARNSAFGFQGRLLHLSAGQ | (G4S)4, G4T, |
| | (SCT) | RLGVHLHTEARARHAWQLTQGATVLGLFRVTPEIPAGGGGSGGGGSGGGGSGGGGSGGGGSGGG | (G4S)2 linker 2 |
| | | GSGGGGSDPAGLLDLRQGMFAQLVAQNVLLIDGPLSWYSDPGLAGVSLTGGLSYKEDTKELVVAK | |
| | | AGVYYVFFQLELRRVVAGEGSGSVSLALHLQPLRSAAGAAALALTVDLPPASSEARNSAFGFQGRL | |
| | | LHLSAGQRLGVHLHTEARARHAWQLTQGATVLGLFRVTPEIPAGGGGSGGGGSGGGGSGGGGSGG | |
| | | GGTGGGGSGGGGSDPAGLLDLRQGMFAQLVAQNVLLIDGPLSWYSDPGLAGVSLTGGLSYKEDTK | |
| | | ELVVAKAGVYYVFFQLELRRVVAGEGSGSVSLALHLQPLRSAAGAAALALTVDLPPASSEARNSAF | |
| | | GFQGRLLHLSAGQRLGVHLHTEARARHAWQLTQGATVLGLFRVTPEIPAS | |
| SEQ ID NO: 8 | GITR | QRPTGGPGCGPGRLLLGTGTDARCCRVHTTRCCRDYPGEECCSEWDCMCVQPEFHCGDPCCTTCR | Q9Y5U5 |
| | | HHPCPPGQGVQSQGKFSFGFQCIDCASGTFSGGHEGHCKPWTDCTQFGFLTVFPGNKTHNAVCVPG | |
| | | SPPAEP | |
| SEQ ID NO: 9 | CD27 | ATPAPKSCPERHYWAQGKLCCQMCEPGTFLVKDCDQHRKAAQCDPCIPGVSFSPDHHTRPHCESCR | P26842 |
| | | HCNSGLLVRNCTITANAECACRNGWQCRDKECTECDPLPNPSLTARSSQALSPHPQPTHLPYVSEML | |
| | | EARTAGHMQTLADFRQLPARTLSTHWPPQRSLCSSDFIR | |
| SEQ ID NO: 10 | B7-1 (CD80) | VIHVTKEVKEVATLSCGHNVSVEELAQTRIYWQKEKKMVLTMMSGDMNIWPEYKNRTIFDITNNLS | P33681 |
| | | IVILALRPSDEGTYECVVLKYEKDAFKREHLAEVTLSVKADFPTPSISDFEIPTSNIRRIICSTSGGFPEP | |
| | | HLSWLENGEELNAINTTVSQDPETELYAVSSKLDFNMTTNHSFMCLIKYGHLRVNQTFNWNTTKQE | |
| | | HFPDN | |
| SEQ ID NO: 11 | B7-2 (CD86) | APLKIQAYFNETADLPCQFANSQNQSLSELVVFWQDQENLVLNEVYLGKEKFDSVHSKYMGRTSFD | P42081 |
| | | SDSWTLRLHNLQIKDKGLYQCIIHHKKPTGMIRIHQMNSELSVLANFSQPEIVPISNITENVYINLTCSS | |
| | | IHGYPEPKKMSVLLRTKNSTIEYDGVMQKSQDNVTELYDVSISLSVSFPDVTSNMTIFCILETDKTRL | |
| | | LSSPFSIELEDPQPPPDHIP | |
| SEQ ID NO: 12 | ICOS-L | DTQEKEVRAMVGSDVELSCACPEGSRFDLNDVYVYWQTSESKTVVTYHIPQNSSLENVDSRYRNR | O75144 |
| | | ALMSPAGMLRGDFSLRLFNVTPQDEQKFHCLVLSQSLGFQEVLSVEVTLHVAANFSVPVVSAPHSPS | |
| | | QDELTFTCTSINGYPRPNVYWINKTDNSLLDQALQNDTVFLNMRGLYDVVSVLRIARTPSVNIGCCI | |
| | | ENVLLQQNLTVGSQTGNDIGERDKITENPVSTGEKNAAT | |
| SEQ ID NO: 13 | OX40-L | LHCVGDTYPSNDRCCHECRPGNGMVSRCSRSQNTVCRPCGPGFYNDVVSSKPCKPCTWCNLRSGSE | P43489 |
| | | RKQLCTATQDTVCRCRAGTQPLDSYKPGVDCAPCPPGHFSPGDNQACKPWTNCTLAGKHTLQPAS | |
| | | NSSDAICEDRDPPATQPQETQGPPARPITVQPTEAWPRTSQGPSTRPVEVPGGRA | |
| SEQ ID NO: 14 | PDL-1 | FTVTVPKDLYVVEYGSNMTIECKFPVEKQLDLAALIVYWEMEDKNIIQFVHGEEDLKVQHSSYRQR | Q9NZQ7 |
| | | ARLLKDQLSLGNAALQITDVKLQDAGVYRCMISYGGADYKRITVKVNAPYNKINQRILVVDPVTSE | |
| | | HELTCQAEGYPKAEVIWTSSDHQVLSGKTTTTNSKREEKLFNVTSTLRINTTTNEIFYCTFRRLDPEE | |
| | | NHTAELVIPELPLAHPPNER | |
| SEQ ID NO: 15 | FasL | QLFHLQKELAELRESTSQMHTASSLEKQIGHPSPPPEKKELRKVAHLTGKSNSRSMPLEWEDTYGIV | P48023 |
| | | LLSGVKYKKGGLVINETGLYFVYSKVYFRGQSCNNLPLSHKVYMRNSKYPQDLVMMEGKMMSYC | |
| | | TTGQMWARSSYLGAVFNLTSADHLYVNVSELSLVNFEESQTFFGLYKL | |
| SEQ ID NO: 16 | PDL2 | LFTVTVPKELYIIEHGSNVTLECNFDTGSHVNLGAITASLQKVENDTSPHRERATLLEEQLPLGKASF | Q9BQ51 |
| | | HIPQVQVRDEGQYQCIIIYGVAWDYKYLTLKVKASYRKINTHILKVPETDEVELTCQATGYPLAEVS | |
| | | WPNVSVPANTSHSRTPEGLYQVTSVLRLKPPPGRNFSCVFWNTHVRELTLASIDLQSQMEPRTHPT | |
| SEQ ID NO: 17 | CD7 | AQEVQQSPHCTTVPVGASVNITCSTSGGLRGIYLRQLGPQPQDIIYYEDGVVPTTDRRFRGRIDFSGS | P09564 |
| | | QDNLTITMHRLQLSDTGTYTCQAITEVNVYGSGTLVLVTEEQSQGWHRCSDAPPRASALPAPPTGSA | |
| | | LPDPQTASALPDPPAASALP | |
| SEQ ID NO: 18 | CD30L | FPQDRPFEDTCHGNPSHYYDKAVRRCCYRCPMGLFPTQQCPQRPTDCRKQCEPDYYLDEADRCTA | P28908 |
| | | CVTCSRDDLVEKTPCAWNSSRVCECRPGMFCSTSAVNSCARCFFHSVCPAGMIVKFPGTAQKNTVC | |
| | | EPASPGVSPACASPENCKEPSSGTIPQAKPTPVSPATSSASTMPVRGGTRLAQEAASKLTRAPDSPSSV | |
| | | GRPSSDPGLSPTQPCPEGSGDCRKQCEPDYYLDEAGRCTACVSCSRDDLVEKTPCAWNSSRTCECRP | |
| | | GMICATSATNSCARCVPYPICAAETVTKPQDMAEKDTTFEAPPLGTQPDCNPTPENGEAPASTSPTQS | |
| | | LLVDSQASKTLPIPTSAPVALSSTGK | |
| SEQ ID NO: 19 | CD40L | HRRLDKIEDERNLHEDFVFMKTIQRCNTGERSLSLLNCEEIKSQFEGFVKDIMLNKEETKKENSFEM | P29965 |
| | | QKGDQNPQIAAHVISEASSKTTSVLQWAEKGYYTMSNNLVTLENGKQLTVKRQGLYYIYAQVTFCS | |
| | | NREASSQAPFIASLCLKSPGRFERILLRAANTHSSAKPCGQQSIHLGGVFELQPGASVFVNVTDPSQV | |
| | | SHGTGFTSFGLLKL | |
| SEQ ID NO: 20 | CD70 | HRRLDKIEDERNLHEDFVFMKTIQRCNTGERSLSLLNCEEIKSQFEGFVKDIMLNKEETKKENSFEM | P32970 |
| | | QKGDQNPQIAAHVISEASSKTTSVLQWAEKGYYTMSNNLVTLENGKQLTVKRQGLYYIYAQVTFCS | |
| | | NREASSQAPFIASLCLKSPGRFERILLRAANTHSSAKPCGQQSIHLGGVFELQPGASVFVNVTDPSQV | |
| | | SHGTGFTSFGLLKL | |
| SEQ ID NO: 21 | CD83 | TPEVKVACSEDVDLPCTAPWDPQVPYTVSWVKLLEGGEERMETPQEDHLRGQHYHQKGQNGSFD | Q01151 |
| | | APNERPYSLKIRNTTSCNSGTYRCTLQDPDGQRNLSGKVILRVTGCPAQRKEETFKKYRAE | |
| SEQ ID NO: 22 | HLA-G | GSHSMRYFSAAVSRPGRGEPRFIAMGYVDDTQFVRFDSDSACPRMEPRAPWVEQEGPEYWEEETR | P17693 |
| | | NTKAHAQTDRMNLQTLRGYYNQSEASSHTLQWMIGCDLGSDGRLLRGYEQYAYDGKDYLALNED | |
| | | LRSWTAADTAAQISKRKCEAANVAEQRRAYLEGTCVEWLHRYLENGKEMLQRADPPKTHVTHHP | |
| | | VFDYEATLRCWALGFYPAEIILTWQRDGEDQTQDVELVETRPAGDGTFQKWAAVVVPSGEEQRYT | |
| | | CHVQHEGLPEPLMLRWKQSSLPTIPI | |
| SEQ ID NO: 23 | MICA | EPHSLRYNLTVLSWDGSVQSGFLTEVHLDGQPFLRCDRQKCRAKPQGQWAEDVLGNKTWDRETR | Q29983 |
| | | DLTGNGKDLRMTLAHIKDQKEGLHSLQEIRVCEIHEDNSTRSSQHFYYDGELFLSQNLETKEWTMP | |
| | | QSSRAQTLAMNVRNFLKEDAMKTKTHYHAMHADCLQELRRYLKSGVVLRRTVPPMVNVTRSEAS | |
| | | EGNITVTCRASGFYPWNITLSWRQDGVSLSHDTQQWGDVLPDGNGTYQTWVATRICQGEEQRFTC | |
| | | YMEHSGNHSTHPVPSGKVLVLQSHW | |
| SEQ ID NO: 24 | MICB | AEPHSLRYNLMVLSQDESVQSGFLAEGHLDGQPFLRYDRQKRRAKPQGQWAEDVLGAKTWDTET | Q29980 |
| | | EDLTENGQDLRRTLTHIKDQKGGLHSLQEIRVCEIHEDSSTRGSRHFYYDGELFLSQNLETQESTVPQ | |
| | | SSRAQTLAMNVTNFWKEDAMKTKTHYRAMQADCLQKLQRYLKSGVAIRRTVPPMVNVTCSEVSE | |
| | | GNITVTCRASSFYPRNITLTWRQDGVSLSHNTQQWGDVLPDGNGTYQTWVATRIRQGEEQRFTCY | |
| | | MEHSGNHGTHPVPSGKVLVLQSQRTD | |
| SEQ ID NO: 25 | HVEM | LQLHWRLGEMVTRLPDGPAGSWEQLIQERRSHEVNPAAHLTGANSSLTGSGGPLLWETQLGLAFLR | O43557 |
| | | GLSYHDGALVVTKAGYYYIYSKVQLGGVGCPLGLASTITHGLYKRTPRYPEELELLVSQQSPCGRA | |
| | | TSSSRVWWDSSFLGGVVHLEAGEKVVVRVLDERLVRLRDGTRSYFGAFMV | |
| SEQ ID NO: 26 | TNFRSF3 | QAVPPYASENQTCRDQEKEYYEPQHRICCSRCPPGTYVSAKCSRIRDTVCATCAENSYNEHWNYLTI | P36941 |
| | | CQLCRPCDPVMGLEEIAPCTSKRKTQCRCQPGMFCAAWALECTHCELLSDCPPGTEAELKDEVGKG | |
| | | NNHCVPCKAGHFQNTSSPSARCQPHTRCENQGLVEAAPGTAQSDTTCKNPLEPLPPEMSGTMLM | |
| SEQ ID NO: 27 | TNFRSF6B | VAETPTYPWRDAETGERLVCAQCPPGTFVQRPCRRDSPTTCGPCPPRHYTQFWNYLERCRYCNVLC | O95407 |
| | | GEREEEARACHATHNRACRCRTGFFAHAGFCLEHASCPPGAGVIAPGTPSQNTQCQPCPPGTFSASS | |
| | | SSSEQCQPHRNCTALGLALNVPGSSSHDTLCTSCTGFPLSTRVPGAEECERAVIDFVAFQDISIKRLQR | |
| | | LLQALEAPEGWGPTPRAGRAALQLKLRRRLTELLGAQDGALLVRLLQALRVARMPGLERSVRERFL | |
| | | PVH | |
| SEQ ID NO: 28 | ILT3 | QAGPLPKPTLWAEPGSVISWGNSVTIWCQGTLEAREYRLDKEESPAPWDRQNPLEPKNKARFSIPSM | Q8NHJ6 |
| | | TEDYAGRYRCYYRSPVGWSQPSDPLELVMTGAYSKPTLSALPSPLVTSGKSVTLLCQSRSPMDTFLL | |
| | | IKERAAHPLLHLRSEHGAQQHQAEFPMSPVTSVHGGTYRCFSSHGFSHYLLSHPSDPLELIVSGSLED | |
| | | PRPSPTRSVSTAAGPEDQPLMPTGSVPHSGLRRHWE | |
| SEQ ID NO: 29 | ILT4 | QTGTIPKPTLWAEPDSVITQGSPVTLSCQGSLEAQEYRLYREKKSASWITRIRPELVKNGQFHIPSITW | Q8N423 |
| | | EHTGRYGCQYYSRARWSELSDPLVLVMTGAYPKPTLSAQPSPVVTSGGRVTLQCESQVAFGGFILC | |
| | | KEGEEEHPQCLNSQPHARGSSRAIFSVGPVSPNRRWSHRCYGYDLNSPYVWSSPSDLLELLVPGVSK | |
| | | KPSLSVQPGPVVAPGESLTLQCVSDVGYDRFVLYKEGERDLRQLPGRQPQAGLSQANFTLGPVSRS | |
| | | YGGQYRCYGAHNLSSECSAPSDPLDILITGQIRGTPFISVQPGPTVASGENVTLLCQSWRQFHTFLLT | |
| | | KAGAADAPLRLRSIHEYPKYQAEFPMSPVTSAHAGTYRCYGSLNSDPYLLSHPSEPLELVVSGPSMG | |
| | | SSPPPTGPISTPAGPEDQPLTPTGSDPQSGLGRHLGV | |
| SEQ ID NO: 30 | CTL4 | MGGKQRDEDDEAYGKPVKYDPSFRGPIKNRSCTDVICCVLFLLFILGYIVVGIVAWLYGDPRQVLYP | Q53GD3 |
| | | RNSTGAYCGMGENKDKPYLLYFNIFSCILSSNIISVAENGLQCPTPQVCVSSCPEDPWTVGKNEFSQT | |
| | | VGEVFYTKNRNFCLPGVPWNMTVITSLQQELCPSFLLPSAPALGRCFPWTNVTPPALPGITNDTTIQQ | |
| | | GISGLIDSLNARDISVKIFEDFAQSWYWILVALGVALVLSLLFILLLRLVAGPLVLVLILGVLGVLAYG | |
| | | IYYCWEEYRVLRDKGASISQLGFTTNLSAYQSVQETWLAALIVLAVLEAILLLMLIFLRQRIRIAIALL | |
| | | KEASKAVGQMMSTMFYPLVTFVLLLICIAYWAMTALYLATSGQPQYVLWASNISSPGCEKVPINTS | |
| | | CNPTAHLVNSSCPGLMCVFQGYSSKGLIQRSVFNLQIYGVLGLFWTLNWVLALGQCVLAGAFASFY | |
| | | WAFHKPQDIPTFPLISAFIRTLRYHTGSLAFGALILTLVQIARVILEYIDHKLRGVQNPVARCIMCCFK | |
| | | CCLWCLEKFIKFLNRNAYIMIAIYGKNFCVSAKNAFMLLMRNIVRVVVLDKVTDLLLFFGKLLVVG | |
| | | GVGVLSFFFFSGRIPGLGKDFKSPHLNYYWLPIMTSILGAYVIASGFFSVFGMCVDTLFLCFLEDLER | |
| | | NNGSLDRPYYMSKSLLKILGKKNEAPPDNKKRKK | |
| SEQ ID NO: 31 | Tim3 | MFSHLPFDCVLLLLLLLLTRSSEVEYRAEVGQNAYLPCFYTPAAPGNLVPVCWGKGACPVFECGNV | Q8TDQ0 |
| | | VLRTDERDVNYWTSRYWLNGDFRKGDVSLTIENVTLADSGIYCCRIQIPGIMNDEKFNLKLVIKPAK | |
| | | VTPAPTRQRDFTAAFPRMLTTRGHGPAETQTLGSLPDINLTQISTLANELRDSRLANDLRDSGATIRI | |
| | | GIYIGAGICAGLALALIFGALIFKWYSHSKEKIQNLSLISLANLPPSGLANAVAEGIRSEENIYTIEENV | |
| | | YEVEEPNEYYCYVSSRQQPSQPLGCRFAMP | |

1. (canceled)

2. (canceled)

3. (canceled)

4. (canceled)

5. (canceled)

6. (canceled)

7. (canceled)

8. (canceled)

9. A fusion polypeptide comprising a single chain variable (scFv) domain, wherein the scFv domain includes:
at least one targeting domain that targets a portion of a myc epitope; and
a CD28 polypeptide antibody, fragment, or derivative thereof or a 4-1BBL polypeptide antibody, fragment, or derivative thereof.

10. A composition comprising the fusion polypeptide according to claim 9 and a pharmaceutically acceptable excipient.

11. The composition of claim 10, further comprising a chimeric antigen receptor (CAR)-expressing cell, wherein the CAR-expressing cell comprises a targeted moiety that is recognized by the targeting moiety of the fusion polypeptide.

12. (canceled)

13. The composition according to claim 11 for use in in treating and/or preventing a disease or condition in a subject.

14. (canceled)

15. (canceled)

16. (canceled)

17. (canceled)

18. (canceled)

19. (canceled)

20. (canceled)

21. (canceled)

22. (canceled)

23. A nucleic acid comprising a nucleotide sequence encoding a fusion polypeptide, wherein the fusion polypeptide comprises a single chain variable (scFv) domain, wherein the scFv domain includes:
at least one targeting domain that targets a portion of a myc epitope; and
a CD28 polypeptide antibody, fragment, or derivative thereof or a 4-1BBL polypeptide antibody, fragment, or derivative thereof.

24. A composition comprising the nucleic acid according to claim 23 and a pharmaceutically acceptable excipient.

25. (canceled)

26. An in vivo method of modulating a CAR-expressing cell, comprising administering to a subject the composition according to of claim 13, wherein the CAR-expressing cell comprises a targeted moiety that is recognized by the targeting moiety of the fusion polypeptide.

27. The method of claim 26, wherein modulation of the CAR-expressing cell induces proliferation of the CAR-expressing cell, induces differentiation of the CAR-expressing cell, stimulates the CAR-expressing cell, inhibits the CAR-expressing cell, or any combination thereof.

28. The method of claim 27, wherein the modulation of the CAR-expressing cell is via direct targeting of the CAR-expressing cell.

29. The method according to claim 28, wherein the modulation of the CAR-expressing cell is at least about 0.5-fold higher as compared to modulation of a CAR-expressing cell without the epitope that is recognized by the targeting domain of the fusion polypeptide.

30. A method of treating a subject, comprising administering to the subject a therapeutically effective amount of the composition according to claim 13.

31. The method of claim 30, wherein the subject is further administered a CAR-expressing cell, wherein the CAR-expressing cell comprises a targeted moiety that is recognized by the targeting moiety of the fusion polypeptide, and wherein the CAR-expressing cell is administered prior to, subsequent to, or concurrently with the fusion polypeptide.

32. (canceled)

33. The method of claim 30, wherein the subject has a disease associated with expression of a tumor antigen or a disease associated with a pathogen, wherein the disease associated with expression of a tumor antigen is a proliferative disease, a precancerous condition, a cancer, or a non-cancer related indication associated with expression of the tumor antigen.

34. (canceled)

35. The method of claim 30, wherein the cancer is chronic lymphocytic leukemia (CLL), acute leukemia, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia, or wherein the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of the cancers, and metastatic lesions of the cancers.

36. (canceled)

37. The method of claim 30, wherein the cancer is leukemia or lymphoma, and wherein the lymphoma is lymphoblastic lymphoma or B-cell Non-Hodgkin's lymphoma.

38. (canceled)

39. (canceled)

40. (canceled)

41. The method of claim 31, comprising modulation of the CAR-expressing cell, the modulation further comprising inducing proliferation of the CAR-expressing cell, inducing differentiation of the CAR-expressing cell, stimulating the CAR-expressing cell, inhibiting the CAR-expressing cell, or any combination thereof.

42. (canceled)

43. The method of claim 42, wherein the modulation of the CAR-expressing cell is via direct targeting of the CAR-expressing cell.

44. The method according to claim 43, wherein the modulation of the CAR-expressing cell is at least about 0.5-fold higher as compared to modulation of a CAR-expressing cell without the targeted moiety that is recognized by the targeting domain of the fusion polypeptide.

45. The method according to claim 44, wherein the subject experiences an improvement in an inflammatory response, a cytokine storm, or at least one off-target effect, or any combination thereof as compared to a control subject.

46. The method according to claim 45, wherein the subject experiences an improvement in a symptom or a side effect as compared to a control subject, and wherein the symptom is cytokine-release syndrome (CRS), a neurologic toxicity, B-cell aplasia, tumor lysis syndrome (TLS), anaphylaxis, fever, joint/muscle aches, shortness of breath, low blood pressure, confusion, a seizure.

47. (canceled)


SEQUENCE LISTING (31 sequences)

SEQ ID NO: 1 - protein, 10 aa, Artificial Sequence
  Myc
EQKLISEEDL

SEQ ID NO: 2 - protein, 7 aa, Artificial Sequence
  FLAG
DYKDDDK

SEQ ID NO: 3 - protein, 5 aa, Artificial Sequence
  His5
HHHHH

SEQ ID NO: 4 - protein, 6 aa, Artificial Sequence
  His6
HHHHHH

SEQ ID NO: 5 - protein, 9 aa, Artificial Sequence
  Hemagglutinin (HA)
YPYDVPDYA

SEQ ID NO: 6 - protein, 247 aa, Artificial Sequence
  Bispecific anti-CD28 scFv
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKRGGGGSGGGGSGGGGSQVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSS

SEQ ID NO: 7 - protein, 572 aa, Artificial Sequence
  41BBL single chain trimer (SCT)
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGGGGSGGGGSGGGGSGGGGSGGGGSGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGGGGSGGGGSGGGGSGGGGSGGGGTGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAS

SEQ ID NO: 8 - protein, 137 aa, Homo sapiens
QRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEP

SEQ ID NO: 9 - protein, 172 aa, Homo sapiens
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 10 - protein, 208 aa, Homo sapiens [sequence omitted]

SEQ ID NO: 11 - protein, 224 aa, Homo sapiens
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 12 - protein, 238 aa, Homo sapiens [sequence omitted]

SEQ ID NO: 13 - protein, 186 aa, Homo sapiens [sequence omitted]

SEQ ID NO: 14 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 15 - protein, 179 aa, Homo sapiens
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 16 - protein, 201 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 17 - protein, 155 aa, Homo sapiens
AQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALP

SEQ ID NO: 18 - protein, 361 aa, Homo sapiens
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGK

SEQ ID NO: 19 - protein, 215 aa, Homo sapiens
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 20 - protein, 215 aa, Homo sapiens; identical to SEQ ID NO: 19

SEQ ID NO: 21 - protein, 125 aa, Homo sapiens
TPEVKVACSEDVDLPCTAPWDPQVPYTVSWVKLLEGGEERMETPQEDHLRGQHYHQKGQNGSFDAPNERPYSLKIRNTTSCNSGTYRCTLQDPDGQRNLSGKVILRVTGCPAQRKEETFKKYRAE

SEQ ID NO: 22 - protein, 284 aa, Homo sapiens [sequence omitted]

SEQ ID NO: 23 - protein, 284 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 24 - protein, 287 aa, Homo sapiens
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTD

SEQ ID NO: 25 - protein, 182 aa, Homo sapiens
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 26 - protein, 197 aa, Homo sapiens
QAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLM

SEQ ID NO: 27 - protein, 271 aa, Homo sapiens
VAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 28 - protein, 238 aa, Homo sapiens
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWE

SEQ ID NO: 29 - protein, 440 aa, Homo sapiens
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGV

SEQ ID NO: 30 - protein, 710 aa, Homo sapiens
MGGKQRDEDDEAYGKPVKYDPSFRGPIKNRSCTDVICCVLFLLFILGYIVVGIVAWLYGDPRQVLYPRNSTGAYCGMGENKDKPYLLYFNIFSCILSSNIISVAENGLQCPTPQVCVSSCPEDPWTVGKNEFSQTVGEVFYTKNRNFCLPGVPWNMTVITSLQQELCPSFLLPSAPALGRCFPWTNVTPPALPGITNDTTIQQGISGLIDSLNARDISVKIFEDFAQSWYWILVALGVALVLSLLFILLLRLVAGPLVLVLILGVLGVLAYGIYYCWEEYRVLRDKGASISQLGFTTNLSAYQSVQETWLAALIVLAVLEAILLLMLIFLRQRIRIAIALLKEASKAVGQMMSTMFYPLVTFVLLLICIAYWAMTALYLATSGQPQYVLWASNISSPGCEKVPINTSCNPTAHLVNSSCPGLMCVFQGYSSKGLIQRSVFNLQIYGVLGLFWTLNWVLALGQCVLAGAFASFYWAFHKPQDIPTFPLISAFIRTLRYHTGSLAFGALILTLVQIARVILEYIDHKLRGVQNPVARCIMCCFKCCLWCLEKFIKFLNRNAYIMIAIYGKNFCVSAKNAFMLLMRNIVRVVVLDKVTDLLLFFGKLLVVGGVGVLSFFFFSGRIPGLGKDFKSPHLNYYWLPIMTSILGAYVIASGFFSVFGMCVDTLFLCFLEDLERNNGSLDRPYYMSKSLLKILGKKNEAPPDNKKRKK

SEQ ID NO: 31 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP